(19) United States
(12) Patent Application Publication
(10) Pub. No.: US 2017/0304369 A1
(43) Pub. Date: Oct. 26, 2017

(54) METHODS OF DIFFERENTIATING STEM CELLS INTO LIVER CELL LINEAGES

(71) Applicant: Agency for Science, Technology and Research, Singapore (SG)

(72) Inventors: Lay Teng ANG, Singapore (SG); Kyle M. LOH, Singapore (SG); Bing LIM, Singapore (SG)

(21) Appl. No.: 15/517,912

(22) PCT Filed: Oct. 8, 2015
(86) PCT No.: PCT/SG2015/050381

§ 371 Date: Apr. 7, 2017

(30) Foreign Application Priority Data
Oct. 8, 2014 (SG) 10201406436U

(51) Int. Cl. A61K 35/407; C12N 5/071; C12N 5/073; G01N 33/00; C12N 5/00

(52) U.S. Cl. CPC A61K 35/407; C12N 5/0672; C12N 5/0603; C12N 5/00; C12N 2501/00; G01N 33/00

(57) ABSTRACT

The present disclosure provides methods and kits for the differentiation of stem cells into relevant liver cell lineages, as well as methods of using the relevant liver cell lineages in screening for a cellular response, a phenotype and in the treatment of a condition. In one embodiment, stem cells are first differentiated into cells of the definitive endoderm lineage, which are differentiated into posterior foregut (PFG) lineage cells by one or more of retinoic acid activators and/or one or more inhibitors of transforming growth factor-β (TGFβ). An additional embodiment provides a method for the differentiation of posterior foregut lineage cells into liver bud progenitors (LB) by one or more activators of TGFβ signalling, and/or one or more modulators of Wnt signalling, and/or one or more activators of cyclic AMP/PKA signaling; and a further embodiment provides a method for the differentiation of liver bud progenitors into hepatic progenitors by one or more inhibitors of TGFβ signalling and/or fibroblast growth factor (FGF) inhibitors and/or one or more Notch inhibitors. Another embodiment discloses the differentiation of hepatic progenitors into hepatocyte-like cells or perivenous hepatocyte-like cells by one or more of Notch inhibitors and/or activators of glucocorticoid signalling and/or one or more activators of insulin signalling and/or one or more of ascorbic acid signalling activators and/or additional factors. Methods and kits for maintaining LB in self renewal state, hepatocyte-like cells in perivenous or periportal state, as well as surface markers for LB and mid/hindgut (MHG) cells are also disclosed.

### CROSS-REFERENCE TO RELATED APPLICATIONS

This application claims the benefit of priority of Singapore application No. 10201406436U, filed 8 Oct. 2014, the contents of it being hereby incorporated by reference in its entirety for all purposes.

### FIELD OF THE INVENTION

The present invention relates generally to the field of biotechnology. In particular, the present invention relates to methods for differentiating cells of the definitive endoderm into multiple cell lineages. The present invention further relates to kits and culture media for use in performing the methods as described herein.

### BACKGROUND OF THE INVENTION

End-stage liver failure results in severe clinical symptoms including bleeding, encephalopathy and eventually death. Notably, the liver contains predominantly hepatocytes, which execute a diverse range of functions vital to the living organism. These functions include the elimination of harmful toxic byproducts in the bloodstream, such as tyrosine metabolites and ammonia and the secretion of serum proteins, including albumin and coagulation factors.

The development and morphogenesis of the liver is a complex process. During early embryonic development, pluripotent epiblast cells form the anterior primitive streak at ˜E6.5, which then gives rise to a sheet of cells known as the endodermal germ layer at ˜E7.5. At E8.5, the endoderm extends along the anterior posterior axis of the embryo and closes up to form a gut tube, eventually giving rise to organs along the entire respiratory and digestive tract. During gut tube formation, spatially distinct lateral and medial endoderm progenitors converge at the midline to form posterior-foregut (PFG) and subsequently the liver bud (LB), as shown by fate mapping studies in the mouse embryos.

There have been instrumental studies performed on the early development of the liver, however, understanding of the processes including liver specification, formation of liver cells and maturation of liver functions, remains limited. For instance, mechanisms that induce formation of liver stem cells as well as their mature progeny remain unclear.

Furthermore, factors endowing liver cells the ability to engraft, proliferate and differentiate in vivo remain to be fully examined, wherein the difficulty in attaining adult-like liver cells is still widely experienced.

Altogether, although pluripotent stem cells could yield liver cells with some liver function, there remains an apparently un-surmounted barrier for these liver cells to progress into an adult-like state. Furthermore, generating ‘authentic’ and transplantable hepatocyte-like cells from these pluripotent stem cells that could engraft and robustly repopulate in the adult liver remains challenging.

In view of these challenges, it is important to understand the signaling logic underlying multiple steps of induction and patterning of the germ layers and differentiation into the various cell lineages. In particular, it is important to understand the signaling factors that regulate liver specification, and to formulate a signaling paradigm for hepatogenesis using human pluripotent stem cell-based differentiation.

It is an aim of the present invention to elucidate the underlying signaling logic of stem cell induction and differentiation in order to unilaterally drive stem cells to generate liver cells with minimal extraneous lineages.

### SUMMARY OF THE INVENTION

According to one aspect, there is provided a method of differentiating cells of the definitive endoderm (DE) lineage into posterior foregut (PFG) lineage comprising contacting said stem cells with: one or more retinoic acid activators; and one or more inhibitors of TGFβ signaling.

According to another, aspect there is provided a method of differentiating cells of the posterior foregut lineage into liver bud (LB) progenitors comprising contacting said cells of the posterior foregut lineage with one or more activators of TGFβ signaling, one or more modulators of Wnt signaling; and one or more activators of cyclic AMP/PKA signaling.

According to another aspect, there is provided a method of differentiating liver bud progenitors into hepatic progenitors comprising contacting said liver bud progenitors with: one or more inhibitors of TGFβ signaling; one or more inhibitors of FGF signaling; and one or more inhibitors of Notch signaling.

According to another aspect, there is provided a method of differentiating hepatic progenitors into perivenous hepatocytes comprising contacting said hepatic progenitors with: one or more inhibitors of Notch signaling, one or more activators of glucocorticoid signaling, one or more activators of insulin signaling; one or more activators of ascorbic acid signaling, and one or more activators of TGFβ signaling.

According to another aspect, there is provided a method of differentiating hepatic progenitors into hepatocyte-like cells comprising contacting said hepatic progenitors with: one or more activators of cyclic AMP/PKA signaling, one or more activators of glucocorticoid signaling; one or more activators of insulin signaling; one or more activators of ascorbic acid signaling, and one or more inhibitors of Notch signaling.

According to another aspect, there is provided a method of maintaining liver bud progenitors in a self-renewal state comprising contacting said liver bud progenitors with: one or more activators of FGF signaling, and one or more activators of Wnt signaling.

According to another aspect, there is provided a method of maintaining hepatocytes or hepatocyte-like cells in a perivenous state comprising contacting said cells with one or more inhibitors of Notch signaling and/or one or more activators of Wnt signaling.

According to another aspect, there is provided a method of maintaining heaptocytes in a periportal state comprising contacting said cells with one or more activators of cyclic AMP/PKA signaling.

According to another aspect, there is provided a kit for differentiating cells of the definitive endoderm (DE) lineage into posterior foregut lineage comprising one or more retinoic acid activators, and one or more inhibitors of TGFβ signaling.

According to another aspect, there is provided a kit for differentiating cells of the posterior foregut lineage into liver bud progenitors comprising one or more activators of TGFβ signaling; one or more modulators of Wnt signaling; and/or one or more activators of cyclic AMP/PKA signaling.

According to another aspect, there is provided a kit for differentiating liver bud progenitors into hepatic progenitors comprising one or more inhibitors of TGFβ signaling; one or more inhibitors of FGF signaling; and/or one or more inhibitors of Notch signaling.

According to another aspect, there is provided a kit for differentiating liver bud progenitors into hepatic progenitors comprising one or more inhibitors of Notch signaling activators of ascorbic acid signaling; one or more activators of cyclic AMP/PKA signaling; and/or one or more activators of insulin signaling.

According to another aspect, there is provided a kit for differentiating hepatic progenitors into perivenous hepatocyte-like cells comprising one or more inhibitors of Notch signaling; one or more activators of glucocorticoid signaling; one or more activators of insulin signaling; one or more activators of ascorbic acid signaling; and/or one or more activators of TGFβ signaling.

According to another aspect, there is provided a kit for differentiating hepatic progenitors into hepatocytes or hepatocyte-like cells comprising one or more activators of cyclic AMP/PKA signaling; one or more activators of glucocorticoid signaling; one or more activators of insulin signaling; one or more activators of ascorbic acid signaling; and/or one or more inhibitors of Notch signaling.

According to another aspect, there is provided a kit for maintaining liver bud progenitors in a self-renewal state comprising one or more of the following factors: one or more activators of FGF signaling and/or one or more activators of Wnt signaling.

According to another aspect, there is provided a kit for maintaining hepatocytes or hepatocyte-like cells in a perivenous state comprising one or more of the following factors: one or more inhibitors of Notch signaling and/or one or more activators of Wnt signaling.

According to another aspect, there is provided a kit for maintaining hepatocytes or hepatocyte-like cells in a periportal state comprising one or more activators of cyclic AMP/PKA signaling.

According to another aspect, there is provided a surface marker for isolating or selecting for LB cells selected from EGFR or CD99.

According to another aspect, there is provided a surface marker for isolating or selecting for MHG cells comprising CD325 (N-cadherin).

According to another aspect, there is provided a method of screening for a cellular response, the method comprising: a) contacting a population of cells generated according to the methods as described herein with a pharmacological agent; and b) evaluating the population of cells for a cellular response induced by the pharmacological agent.

According to another aspect, there is provided a method of screening for a phenotype, the method comprising: a) administering to a host animal a population of cells generated according to the methods as described herein, wherein the cells of the population of cells comprise a genetic modification in at least one genetic locus; and b) evaluating the host animal for a detectable phenotype induced by the administered population of cells.

According to another aspect, there is provided a method of treating a subject for a condition, the method comprising: a) administering the subject a therapeutically effective amount of cells generated according to the methods described herein in order to treat the subject for the condition.

According to another aspect, there is provided use of a therapeutically effective amount of cells generated according to the methods as described herein in the manufacture of a medicament for treating a condition in a subject.

### DETAILED DESCRIPTION OF THE PRESENT INVENTION

### Definitions

The following words and terms used herein shall have the meaning indicated:

As used herein, the term “stem cells” include but are not limited to undifferentiated cells defined by their ability at the single cell level to both self-renew and differentiate to produce progeny cells, including self-renewing progenitors, non-renewing progenitors, and terminally differentiated cells. For example, “stem cells” may include (1) totipotent stem cells; (2) pluripotent stem cells; (3) multipotent stem cells; (4) oligopotent stem cells; and (5) unipotent stem cells.

As used herein, the term “pluripotent stem cell” (PSC) refers to a cell with the developmental potential, under different conditions, to differentiate to cell types characteristic of all three germ cell layers, i.e., endoderm (e.g., gut tissue), mesoderm (including blood, muscle, and vessels), and ectoderm (such as skin and nerve). The developmental competency of a cell to differentiate to all three germ layers can be determined using, for example, a nude mouse teratoma formation assay. In some embodiments, pluripotency can also be evidenced by the expression of embryonic stem (ES) cell markers, although the preferred test for pluripotency of a cell or population of cells generated using the compositions and methods described herein is the demonstration that a cell has the developmental potential to differentiate into cells of each of the three germ layers.

As used herein, the term “induced pluripotent stem cells” or, iPSCs, means that the stem cells are produced from differentiated adult cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “embryonic stem cell” refers to naturally occurring pluripotent stem cells of the inner cell mass of the embryonic blastocyst. Such cells can similarly be obtained from the inner cell mass of blastocysts derived from somatic cell nuclear transfer. Embryonic stem cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta, i.e., are not totipotent.

As used herein, the term “Differentiation” is the process by which an unspecialized (“uncommitted”) or less specialized cell acquires the features of a specialized cell such as, for example, a nerve cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein, the term “undifferentiated cell” refers to a cell in an undifferentiated state that has the property of “self-renewal” and has the developmental potential to differentiate into multiple cell types, without a specific implied meaning regarding developmental potential (i.e., totipotent, pluripotent, multipotent, etc.). As used herein, the term “self-renewal” or “self renewal state” refers to a stable cellular state whereby cells continue to replicate itself unchangingly so that they retain the potential and competence to form their daughter cell types. The term “competence” in the context of stem cells refers to the capacity or potential of one cell to differentiate into its daughter cell type. For example, the posterior foregut is competent when it may give rise to its daughter cell types such as the pancreatic endoderm and LB cells.

As used herein, the terms “maintenance”, “maintain” and the like refers to preserving aspects or qualities (including but not limited to gene expression) of a progenitor cell over time with or without proliferation of the cell. As used herein, the terms “maintenance”, “maintain” and the like also indicate that the progression of the progenitor to the next developmental stage is stalled, slowed or reduced.

As used herein, the terms “liver”, “hepatic” and the like refer to its conventional meaning appreciated by those skilled in the art, whereby the liver comprises smaller units of liver lobules each comprising hepatocytes in one of two regions, either being near the periportal or perivenous region. The periportal hepatocytes are liver cells that are located near the portal vein in the liver lobule, whereby relevant genes expressed by periportal hepatocytes include but are not limited to carbamoyl-phosphate synthase 1 (CPS), Arginine 1 (ARG1) and phosphoenolpyruvate carboxykinase (PCK1). In contrast, the perivenous hepatocytes are liver cells that are located near the central vein in the liver lobule, whereby relevant genes expressed by perivenous hepatocytes may include but are not limited to Glutamine Synthetase (GS) and Glutamate Transporter (GLT1), and may also incude expression of Fumarylacetoacetate hydrolase (FAH), T-box3 (TBX3) and Cytochrome P450, family 3, subfamily A, (CYP3A4). Moreover, perivenous hepatocytes may also include heterogeneous subtypes such as adult human hepatic stem cells that are responsive to Wnt modulation and that may self-renew under uninjured conditions.

As used herein, the terms “hepatic cells’, “hepatocyte-like cells”, “hepatocytes” and the like refer to cells differentiated from human PSCs that show liver-like qualities such as expression of liver genes and proteins and/or hepatic cells derived or isolated from human livers which possess liver gene/protein expression and liver functions. A number of genes expressed in the liver includes carrier protein Albumin (ALB), blood coagulation factors Fibrinogen Alpha chain (FBA), Fibrinogen Beta chain (FBB), Fibrinogen Gamma chain (FBG), thrombinogen, Alphal anti-trypsin (AAT), Tyrosine metabolic genes [Fumarylacetoacetate hydrolase (FAH), tyrosine amino transferase (TAT), homogentisate 1,2-dioxygenase (HGD), 4-hydroxyphenylpyruvic acid dioxygenase (HPD), phenylalanine hydroxylase (PAH), maleylacetoacetate isomerase (MAI)], and urea metabolic genes (ARG1), ornithine carbamoyltransferase (OTC), Carbamoyl-phosphate synthase 1 (CPS1), Glutamine Synthetase (GS). The term “hepatocytes or hepatocyte-like cells” includes different hepatocyte subtypes, which includes but not limited to stem cells, progenitors, differentiated perivenous hepatocytes, and periportal hepatocytes. Furthermore, the term “hepatocyte-like cells” refers to cells that may possess liver or hepatic stem cell or progenitor properties, including but not limited to the capacity to self renew, proliferate and differentiate. Hepatocyte-like cells may or may not be mature and functional but can engraft into the liver. Moreover, the term “Mature hepatocytes” refer to hepatocytes which express high levels of functional liver genes such as AAT, ALB, FAH, TAT, ARG1, OTC, CYP3A4, GS, GLT1, PAH, HPD, HGD, OTC, FBB, FBA, FBG.

As used herein, the terms “mature”, “maturity” and the like describe the final developmental stage of a cell during the course of differentiation and development. In this regard, the term “more mature” in the context of a cell refers to a cell that is at least one developmental stage more advanced than a “less mature” cell. Further, it will be appreciated that cell(s) in the adult possess the highest level of maturity. The phrase “differentiate towards mature cells” indicates achieving higher level of maturity in differentiated cells during lineage specification, and the expression of “mature” genes refers to expression of genes that are important for the function and phenotype of a functional cell.

As used herein, the term “progenitor cell” refers to cells that have greater developmental potential, i.e., a cellular phenotype that is more primitive (e.g., is at an earlier step along a developmental pathway or progression) relative to a cell which it can give rise to by differentiation. Often, progenitor cells have significant or very high proliferative potential. Progenitor cells can give rise to multiple distinct cells having lower developmental potential, i.e., differentiated cell types, or to a single differentiated cell type, depending on the developmental pathway and on the environment in which the cells develop and differentiate.

As used herein, the term “markers” refers to nucleic acid or polypeptide molecule that is differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

As used herein, the term “modulator” refers to any molecule or compound which either enhances or inhibits the biological activity of the defined signaling pathway or its target. The inhibitors or activators may include but are not limited to peptides, antibodies, or small molecules that target the receptors, transcription factors, signaling mediators/transducers and the like that are a part of the signaling pathway or the targets natural ligand thereby modulating the biological activity of the signaling pathways. In this regard, as used herein “inhibitors” or “activators” refers to the inhibition or activation of one or more components of the defined signaling, including but not limited to the signaling ligands, receptors, transducers, signaling mediators and transcriptional factors. In particular, “inhibitors” or “activators” may refer to antagonists or agonists of the ligand protein of the signaling pathways or any component of the signaling transduction pathways besides the ligand protein, (e.g. the receptors, transducers, signaling mediators)

As used herein the phrases “culture medium”, “differentiation medium” and the like refer to a liquid substance used to support the growth of stem cells and any of the cell lineages. The culture medium used by the invention according to some embodiments can be a liquid-based medium, for example water, which may comprise a combination of substances such as salts, nutrients, minerals, vitamins, amino acids, nucleic acids, proteins such as cytokines, growth factors and hormones.

As used herein, the term “feeder cell” refers to feeder cells (e.g., fibroblasts) that maintain stem cells in a proliferative state when the stem cells are co-cultured on the feeder cells or when the pluripotent stem cells are cultured on a matrix (e.g., an extracellular matrix, a synthetic matrix) in the presence of a conditioned medium generated by the feeder cells. The support of the feeder cells depends on the structure of the feeder cells while in culture (e.g., the three dimensional matrix formed by culturing the feeder cells in a tissue culture plate), function of the feeder cells (e.g., the secretion of growth factors, nutrients and hormones by the feeder cells, the growth rate of the feeder cells, the expansion ability of the feeder cells before senescence) and/or the attachment of the stem cells to the feeder cell layer(s).

As used herein, the terms “treatment”, “treating”, “treat” and the like are used to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with mesodermal cell type dysfunction or deficiency, e.g. those having liver dysfunction or deficiency as well as those in which prevention is desired (e.g., those with increased susceptibility to a liver cell type dysfunction or deficiency; those suspected of having a mesodermal cell type dysfunction or deficiency; those having one or more risk factors for a hepatic cell type, etc.).

As used herein, the term “hepatocyte-like cells” includes but not limited to periportal hepatocytes. “Hepatocyte-like cells” may include perivenous hepatocytes.

As used herein, the terms “basal media” or “basal medium” or the like refer to a medium that contains a carbon source, water, various salts, and a source of amino acids and nitrogen.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF THE DRAWINGS

The invention will be better understood with reference to the detailed description when considered in conjunction with the non-limiting examples and the accompanying drawings, in which:

FIG. 1A shows a timeline of the signaling strategy for hepatic differentiation from human pluripotent stem cells and signaling modulators that may be used to differentiate human pluripotent stem cells/human ESCs towards anterior primitive streak (APS), definitive endoderm (DE), posterior foregut (PFG), liver bud (LB) progenitors, hepatic progenitors (HP), and hepatocyte-like cells (HC). Other mutually exclusive or alternate cell fates such as mid/hindgut (MHG), pancreatic (PAN), Biliary (BIL) and perivenous hepatocyte-like cells (PV) were inhibited or repressed using certain signaling modulators. FIG. 1B shows the morphology of cells during differentiation. Abbreviations: inh—inhibitor, hi—high, lo—low, mid—middle dose, TGFb^{hi}—high dose Activin/Nodal, FGF—FGF activator, WNT^{mid}—moderate dose of Wnt activator, PI3K^{inh}—PI3kinase inhibitor, BMPinh—Bmp signaling inhibitor, RA—All trans Retinoic acid, FGFlo—low dose of FGF, cAMP—cyclic AMP activator, MAPKinh—MAPK inhibitor, TGFb inh—TGFb inhibitor, NOTCHinh—Notch signaling or gamma secretase inhibitor, OSM—oncostatin M, INS—Insulin, AA2P—L-ascorbic acid 2 phosphate.

FIG. 2A shows the high percentages of Sox17-mCherry expressing DE after 2 days of differentiation. FIG. 2B shows the strategy used to test developmental competence of posterior foregut to generate LB cells. FIG. 2C shows the effects of modulating All-trans Retinoic Acid; II. cAMP; III. TGFβ; IV. FGF and V. Wnt on day 3 during foregut specification on the later downstream differentiation to LB cells based on gene expression of liver bud markers such as AFP, CEBPA, HNF1A, HNF6, HNF1B, PROX1, TBX3 and HNF4A. FIG. 2D shows the schematic illustration of AP patterning by retinoic acid and Wnt signaling.

FIG. 3A shows gene expression analysis of the effects of treating PFG cells with I. RA modulators, II cAMP/PKA modulators, III TGFβ modulators and IV. FGF modulators, and, V BMP modulators during days 4-6 on the expression of liver bud markers. FIG. 3B shows the protein expression of HNF4A, TBX3, AFP, CEBPA in hESC-derived LB cells.

FIG. 3C shows the demarcation of gene expression boundaries in respective organ domains such as LB, pancreatic, and intestinal early progenitors. FIG. 3D shows that Activin treatment during LB specification (days 4-6) later enhances expression of ALB on day 13.

FIG. 4A shows the effects of signals controlling liver maturation in vitro. LB cells were treated with signals modulating I. cyclic AMP/PKA; II. Notch; III. Insulin; IV. Ascorbic acid; V. FGF/MAPK and VI. TGFβ on the gene expression to induce formation of hepatic progenitors (12 days post-differentiation) and hepatocyte-like cells (16 days post-differentiation). FIG. 4B shows that inclusion of KOSR in the basal media during days 7-18 of liver differentiation leads to formation of lipid globules in the hPSC-derived hepatocyte-like cells (detected by oil Red O stain) compared to hESCs. FIG. 4C shows the increased and decreased in ALB expression with addition of BMP4 (B) and BMP inhibitor BM3189 respectively.

FIG. 5A shows the schematics of the surface marker screen conducted on human pluripotent stem cells (hPSC), hPSC-derived definitive endoderm (DE), hPSC-derived liver bud cells (LB) and hPSC-derived mid/hindgut (MHG) cells. FIG. 5B shows the flow cytometry histograms of unique expression of CD99 expression on hPSC-derived LB cells but not DE or hPSCs. FIG. 5C shows that EGFR marks uniquely hPSC-derived LB but not hPSC-derived MHG. Conversely, CD325 marks hPSC-derived MHG but not LB.

FIG. 6A shows expression of human ALBUMIN on liver sections obtained from no-cell FRG−/− control, FRG−/− mice livers transplanted with adult human hepatocytes or with hESC-derived liver cells (D18 hPSC-derived liver) I. shows the engraftment and population of mouse liver; II. depicts the spatial localization of hPSC derived liver cells near blood vessels such as portal and central veins; III. shows a graph of the human ALBUMIN detected in the FRG−/− mice serum from mice that are untransplanted (no cell control), or transplanted with either adult human hepatocytes or hPSC-derived liver; and IV shows a graph indicating the reduction of bilirubin level in serum from transplanted liver cells compared to untransplanted (no cell control) FRG−/− mice. FIG. 6B shows the enhancement of survival in mice transplanted with hPSC-derived liver (18 days post-differentiation).

FIG. 7A shows a summary of known liver developmental timing, and gene expression changes in vivo. FIG. 7B shows a summary of signals that may be used to derive liver cells from definitive endoderm. Note that CHIR and FGF is shown to enhance expansion and self-renewal of LB progenitors. FIG. 7C shows the signals promoting hepatic specification (ALB+ cells) or biliary cells (SOX9+ HNF6+ HNF1B+) or signals promoting proliferation. Lines with arrowhead represents positive effect while lines without arrow head represents inhibitory effects. Abbreviations: inh—inhibitor, hi—high, lo—low, mid—middle dose, TGFb^{hi}—high dose Activin/Nodal, FGF—FGF activator, WNT^{mid}—moderate dose of Wnt activator, PI3K^{inh}—PI3kinase inhibitor, BMPinh—Bmp signaling inhibitor, RA—All trans Retinoic acid, FGFlo—low dose of FGF, cAMP—cyclic AMP activator, MAPKinh—MAPK inhibitor, TGFb inh—TGFb inhibitor, NOTCHinh—Notch signaling or gamma secretase inhibitor, OSM—Oncostatin M, INS—Insulin, AA2P—L—ascorbic acid 2 phosphate, SHH—sonic hedgehog activator, EGF—epidermal growth factor

FIG. 8 shows the signals of I. Wnt and II. TGFb modulation that regulate foregut competence on subsequent LB differentiation.

FIG. 9A shows the signals that regulate early pancreatic endoderm specification from foregut during days 4-6. I—TGFb/Activin A, II-BMP—Bmp signaling modulation, and III FGF—FGF signaling modulation. Abbreviations: SB505—TGFb inhibitor, SB505124, ACT10—Activin (10 ng/ml), ACT20—Activin (20 ng/ml), D—Bmp signaling DM3189, B3, B10, B25—BMP4 at doses 3 ng/ml, 10 ng/ml and 25 ng/ml respectively, F10, F20—FGF2 at doses 10 ng/ml and 20 ng/ml respectively, PD—PD0325901, ACDRS—a combination of Activin, C59, DM3189, RA, SANT1, ACPRS—a combination of Activin, C59, PD0325901, RA, SANT1, BR—Combination of BMP4 and RA. FIG. 9B shows the protein expression of PDX1 and FOXA2 in hPSC-derived pancreatic endoderm by immunostaining. FIG. 9C I. shows the effects of TGFb modulation on LB gene expression. Abbreviations: SB505—TGFb inhibitor SB505124, B—Bmp4, A10, A20, A40—Activin Aat doses 10 ng/ml, 20 ng/ml and 40 ng/ml respectively; and II. shows that Wnt signaling activation increases expression of certain liver bud genes. Abbreviations: DKK1—Dickkopf 1, XAV—XAV939, A83—A83-01, W150-WNT3A at dose 150 ng/ml, CHIR3—CHIR99201 at dose 3 uM. FIG. 9D shows a difference in the morphology of hepatocyte-like cells after earlier treatment of CHIR99201 on days 4-6 during LB induction. FIG. 9E shows that early Wnt inhibition later promotes liver expression in hepatocyte-like cells. Abbreviations: XAV—XAV939, CHIR—CHIR 99201, CHIR1,2,3—CHIR 99201 at 1 μM, 2 μM, 3 μM respectively.

FIG. 10A shows the effect of subtracting individual signaling modulators from a signaling cocktail termed “BCDEFV” on liver gene expression. It shows the effect of different combinations of signaling factors abbreviated as B, V, E, F, C, D, wherein B represents BMP4, V represents VEGF, E represents EGF, F represents FGF2, C represents CHIR and D represents DAPT on gene expression markers associated with hepatic progenitor specification (such as AFP, ALB, TBX3) and biliary specification (SOX9). Removal of CHIR or FGF2 from the ‘BCDEFV’ shows significant increase in ALB expression. This indicates that CHIR and FGF2 inhibits ALB expression. Abbreviations: D10—day 10 of differentiation. FIG. 10B shows the signals controlling liver maturation in vitro. For example, bar chart shows Dexamethasone increases ALB expression. Abbreviations: AA—amino acid, 3×—3× higher concentration than the amount of amino acids in IMDM/F12, 10×—10× higher concentration than the amount of amino acids in IMDM/F12, INS—insulin, Base—base media, CC—Choline Chloride, DEX—dexamethasone, DD—DAPT and Dexamethasone, PD173—PD173074, day 6 harvest—hPSC-derived LB control.

FIG. 11 shows a list of cell surface markers expressed on A. hESCs, B. hESCs-derived definitive endoderm, or C. hESC-derived LB. Darkened blocks indicate high percentage of expression (˜100%), clear blocks represent low percentage of expression (˜0).

FIG. 11D shows CD99 expression on LB but not hESCs/hIPSCs or hESC/hIPSC-derived DE.

FIG. 11E shows a venn diagram summarizing the cell type specific surface marker expression on hESCs, hESCs-derived DE cells or hESCs-derived LB cells.

FIG. 12A shows a schematic diagram of zonated heterogeneous hepatocytes depending on its localization in the liver lobule. Periportal hepatocytes are located near the portal vein, while perivenous hepatocytes are located near the central vein. These two hepatocyte subtypes express different genes. FIG. 12B shows the expression of FAH in human liver. FAH expression appears to be in both populations of hepatocytes.

FIG. 13 shows the effect of A. PKA agonists, B. cis-RA and C. Wnt inhibition on periportal versus perivenous gene expression.

FIG. 14A shows the schematic diagram of characterizing hepatocyte metabolic regression in vitro, whereby adult human hepatocytes were grown in vitro and signaling modulators were added to determine if they could reverse the regression. FIG. 14B shows the significant decrease in liver gene expression (CYP2C19, CYP3A4, PXR, CAR, ARG1, PCK1, HNF4A, CEBPA) of hepatocytes during in vitro culture. FIG. 14C shows a summary of the signals that reduce regression of liver maturity in vitro. FIG. 14D shows the effects of modulating i. Notch; ii. TGFβ; iii. Wnt/GSK3B; iv. Estrogen and v. cAMP on gene expression analysis of cytochrome enzymes (CYP1A1, CYP1A2, CYP2C19, CYP2C9, CYP2D6, CYP2E1, CYP3A4, CYP3A5, CYP3A7, CYP7A1) and apical and basal transporters (ABCB11, ABCC2, ABCC3, SLCO1A2).

FIG. 15A shows the effect of modulating cyclic AMP/PKA signaling using 8-bromoCAMP, sp-CAMP, rp-CAMP on liver gene expression such as CYP1, 2, 3 families, BSEP, MRP2, MRP3, AAT, PX, PXR, AAT, PEPCK1, SDH, ALB, FBP1, FBA, FBB, FBG, G6PC. FIG. 15B shows the subtraction screen whereby individual components are removed from a cocktail of signaling factors to determine which component affects liver gene expression. The removal of 8-BromoCAMP results in increased expression of cytochrome genes while the removal of CHIR99201 (CHIR) results in increased expression of periportal genes like ARG1, SERDH, CPS1 and decrease of GLT and GS.

FIG. 16A shows the comparison of commercial media (either Life Technologies, CM4000 base media to grow hepatocytes or XenoTech K2300) and the media disclosed herein (comprising of TGFB signaling inhibitor, Notch signaling inhibitor, PKA inhibitor and wnt activator) on the liver gene expression of adult human primary hepatocytes that were cultured over a time course of days 0, 2, 3, 5, 7 (abbreviated as d0, d2, d3, d5, d7). FIG. 16B bottom panel shows the protein expression of CYP3A4 by immunostaining 5 hours after thawing and 7 days after in vitro culture using commercial media (K2300) or the mHep media disclosed herein.

FIG. 17 shows the effects of foregut and liver induction media on DE cells after 6 total days of differentiation using composition described in Zhao et al., 2012 or Si-Tayeb et al., 2010 or the method disclosed herein, using gene expression analyses. Methods in Zhao et al., 2012 or Si-Tayeb et al., 2010, typically requires a longer period, hence expression of LB genes is higher in cells derived using the method disclosed herein over 6 days. FIG. 17B shows that the addition of HGF at 20 ng/ml during days 13-16 increases the expression of ALB in hepatocyte-like cells.

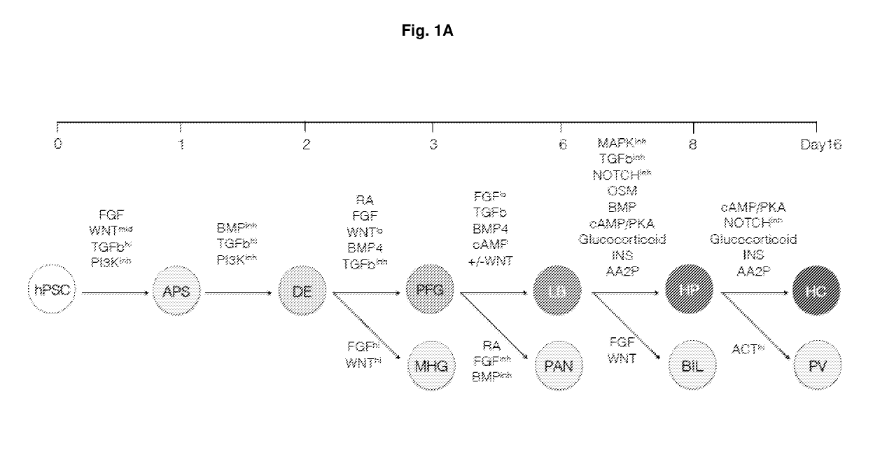

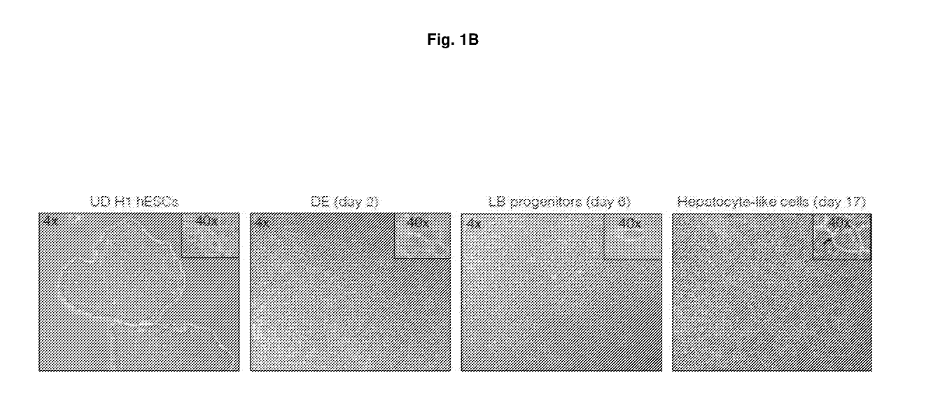

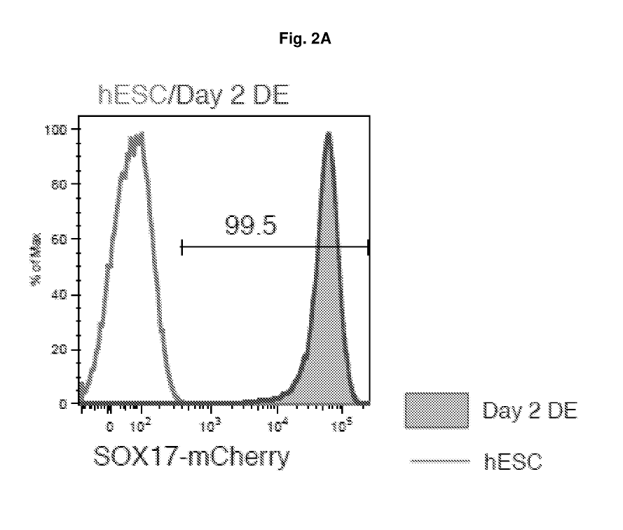

### DESCRIPTION OF EMBODIMENTS

Before the present inventions are described, it is to be understood that this invention is not limited to particular embodiments described, as such may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure. The present disclosure and embodiments relate to the identification of various signaling pathways that regulate different stages of liver development, including foregut and liver bud specification, lineage segregations into hepatic versus biliary fates and finally liver maturation. In particular, the present disclosure and invention exploits definitive endoderm (DE) populations to investigate developmental signals that control anteroposterior and dorso patterning of endoderm into liver bud progenitors, and subsequently identifying factors that drive liver bud progenitors to more differentiated hepatic fates, with the ultimate aim of generating transplantable liver progenitors.

As such, the present disclosure is based upon the further understanding of the signaling mechanisms governing liver development, including its specification and functional attainment. Advantageously, the present disclosure provides the knowledge of signaling controls for different stages of liver differentiation, culminating in the generation of transplantable hepatic progenitors from stem cells and hence allows for the generation of liver cells that may be potentially useful for therapeutic applications.

As such, the present disclosure and embodiments provide methods and cell culture mediums for differentiating pluripotent stem cells into engraftable liver cells. In particular, the present disclosure provides methods of differentiating cells derived from the definitive endoderm (DE) into hepatocyte-like cells through a series of intermediate differentiation steps as outlined in Table 1.

TABLE 1

| | Cell Differentiation | Time |
|---|---|---|
| Step 1 | Definitive endoderm differentiated | About 1 day or may be |
| | into posterior foregut | greater than 1 day |
| Step 2 | posterior foregut differentiated into | About 3 days or may be |
| | liver bud progenitors | greater than 3 days |
| Step 3A | Liver bud progenitors differentiated | About 1-6 days |
| | into hepatic progenitors | |
| Step 3B | Hepatic progenitors from 3A | about 4-6 days or may be |
| | differentiated into hepatic progenitors | greater than 6 days |
| Step 4 | Hepatic progenitors differentiated | about 6 days or may be |
| | into either a) perivenous | greater than 6 days |
| | hepatocyte-like cells; or b) | |
| | hepatocyte-like cells | |

In the context of the present disclosure, the cells of the definitive endoderm may be derived from pluripotent stem cells including but not limited to human embryonic stem cells (hESC), which may or may not be derived from a human embryonic source. As such, the pluripotent stem cells may be human pluripotent stem cells (hPSC).

For example, pluripotent stem cells suitable for use in the present invention may include but are not limited to human embryonic stem cell line H9 (NIH code: WA09), the human embryonic stem cell line H1 (NIH code: WAO1), the human embryonic stem cell line H7 (NIH code: WA07), the human embryonic stem cell line SA002 (Cellartis, Sweden), Hes3 (NIH code: ES03), MeL1 (NIH code: 0139), or stem cells that express at least one of the following markers characteristic of pluripotent cells: ABCG2, cripto, CD9, FoxD3, Connexin43, Connexin45, Oct4, Sox2, Nanog, hTERT, UTF-I, ZFP42, SSEA-3, SSEA-4, Tral-60, Tral-81.

Similarly, the pluripotent stem cell may be an induced pluripotent stem (iPS) cell, which may be derived from non-embryonic sources, and can proliferate without limit and differentiate into each of the three embryonic germ layers. For example an IPS cell line can include but is not limited to BJC1 and BJC3. It is understood that iPS cells behave in culture essentially the same as ESCs.

In this regard, as is well-known in the context of the technical field, pluripotent stem cells may differentiate into functional cells of various cell lineages from the multiple germ layers of either endoderm, mesoderm or ectoderm, as well as to give rise to tissues of multiple germ layers following transplantation and to contribute substantially to most, if not all, tissues following injection into blastocysts.

Accordingly, the definitive endoderm cells may be derived from hPSCs using well known culturing methods to induce definitive endoderm patterning. In particular, such methods to obtain DE cells may yield a heterogeneous mixture of cells, including not only DE cells but also other contaminating lineages. For example, the definitive endoderm cells may be obtained by culturing hPSCs on day 1 with a TGFb activator, a Wnt activator, a PI3K inhibitor, and a FGF activator, and then subsequently on day 2 the cells may be cultured with a TGFb activator, a BMP inhibitor and a PI3k inhibitor. In the methods described herein, the differentiation steps of Table 1 include culturing the cells in a suitable culture medium that is able to support the propagation and/or differentiation of cells into the intended cell lineage. In particular, the culturing of the cells may include the contacting of the cell with one or more of the differentiating factors in vitro. The term “contacting” is not intended to include the in vivo exposure of cells to differentiating factors, and may be conducted in any suitable manner. For example, the cells may be treated in adherent culture, or in suspension culture that include one or more differentiating factors. It is understood that the cells contacted with one or more differentiating factors may be further treated with other cell differentiation environments to stabilize the cells, or to differentiate the cells further.

By measuring expression of particular genes and/or protein markers, progress of differentiation of cells toward the one or more cell lineage may be identified and their progression monitored. Methods for measuring and assessing expression of genes and/or protein markers in cultured or isolated cells are those standard and known in the art. For example, such methods include quantitative reverse transcriptase polymerase chain reaction (RT-PCR), Northern blots, hybridization, ELISA assays, enzymatic activity assays and immunoassays, such as immunohistochemical analysis of sectioned material, immunostaining and fluorescence imaging, Western blotting, and for markers that are accessible in intact cells, flow cytometry analysis (FACS). In particular, isolating lineage specific cells may be effected by sorting of cells via fluorescence activated cell sorter (FACS). In the context of the present disclosure and cell lineages Table 2 outlines genes and/or protein markers that may be used for identifying the differentiation state and lineage of cells.

TABLE 2

| | Marker | |
|---|---|---|
| Cell Lineage | Presence | Absence |
| Definitive Endoderm | SOX17, Surface | Mid/hindgut (MHG) |
| | marker (CXCR 4) | (CDX2), Mesoderm |
| | | (MESP2, MESP1, HAND1, |
| | | FOXF1), ectoderm (PAX6), |
| | | liver or visceral endoderm |
| | | (AFP) |
| posterior foregut | HNF4A | Midgut/hindgut (CDX2) |
| | | expression higher but is also |
| | | expressed in PFG at the |
| | | Foregut/MHG boundary. |
| liver bud | HNF4A, AFP, | Hepatic progenitor (ALB), |
| | TBX3, HNF6, | pancreatic (PDX1), intestinal |
| | CEBPA, HNF1B, | |
| | PROX1 | (CDX2) |
| hepatic progenitors | ALB, AFP, | Biliary (CK19) (cytoplasmic |
| | TBX3 | and surface protein) |
| perivenous hepatocytes | CYP3A4, GS, | Periportal (CPS1) |
| | GLT1 | |
| periportal hepatocytes | CPS1, ARG1 | Perivenous (GS) |
| Hepatocyte-like cells | FAH, TAT, | Lower levels of AFP |
| | HGD, HPD, | |
| | PAH, ALB, | |
| | AAT, FBB, | |
| | FBA, FBG | |

### Differentiation Factors

Various growth factors and other chemical signals may modulate differentiation of stem cells into progeny cell cultures of the one or more particular desired cell lineages. Differentiation factors that may be used in the present invention include but are not limited to compounds or molecules that modulate the activity of one or more of retinoic acid, bone morphogenetic protein (BMP) signaling, transforming growth factor beta (TGFβ) signaling, cyclic AMP/protein kinase A (cyclic AMP/PKA) signaling, growth factor signaling, Wnt Signaling, fibroblast growth factor (FGF) signaling or FGF/mitogen-activated protein kinase (FGF/MAPK) signaling, Notch signaling, protein kinase G (PKG) signaling, Oncostatin M/Gp130 signaling, HGF signaling, steroid hormone signaling, ascorbic acid signaling, vitamin D signaling, L-Glutathione signaling, insulin signaling or glucocorticoid signaling. In addition, the differentiation factors may include but are not limited to amino acid mixtures, or phospholipid precursors.

In one embodiment, the modulators of retinoic acid (RA) may include but are not limited to activators such as retinoic acid precursors, All-trans retinoic acid (ATRA), TTNBP (Arotinoid Acid—4-[(1E)-2-(5,6,7,8-tetrahydro-5,5,8,8-tetramethyl-2-naphthalenyl)-1-propen-1-yl]-benzoic acid), AM580 or vitamin A. The activators of All-trans retinoic acid (ATRA) may include but are not limited to 3,7-Dimethyl-9-(2,6,6-trimethyl-1-cyclohexen-1-yl)-2E,4E,6E,8E,-nonatetraenoic acid; alternative embodiments of ATRA are 9-cis retinoic acid and 13-cis retinoic acid (IUPAC name of 9-cis retinoic acid is 3,7-Dimethyl-9-(2,6,6-trimethyl-1-cyclohexen-1-yl)nona-2E,4E,6Z,8E-tetraenoic acid and 13-cis retinoic acid is (2Z,4E,6E,8E)-3,7-dimethyl-9-(2,6,6-trimethylcyclohexen-1-yl)nona-2,4,6,8-tetraenoic acid).

In one embodiment, the modulators of BMP signaling may include but are not limited to activators such as BMP4, BMP 7 and BMP2.

In one embodiment, the modulators of FGF signaling may include but are not limited to activators such as FGF2, FGF4, FGF8, FGF10 or other family members of the FGF signaling pathway.

In one embodiment, the modulators of FGF/MAPK signaling may include but are not limited to inhibitors such as PD0325901 (N-[(2R)-2,3-Dihydroxypropoxy]-3,4-difluoro-2-[(2-fluoro-4-iodophenyl)amino]-benzamide), PD173074 (N-[2-[[4-(Diethylamino)butyl]amino]-6-(3,5-dimethoxyphenyl)pyrido[2,3-d]pyrimidin-7-yl]-N′-(1,1-dimethylethyl)urea), PD-161570 (N-[6-(2,6-Dichlorophenyl)-2-[[4-(diethylamino)butyl]amino]pyrido [2,3-d]pyrimidin-7-yl]-N′-(1,1-dimethylethyl)urea), FIIN 1 hydrochloride (N-(3-((3-(2,6-dichloro-3,5-dimethoxyphenyl)-7-(4-(diethylamino)butylamino)-2-oxo-3,4—dihydropyrimido[4,5-d]pyrimidin-1(2H)-yl)methyl)phenyl)acrylamide), FR-180204 (5-(2-Phenyl-pyrazolo[1,5-a]pyridin-3-yl)-1H-pyrazolo[3,4-c]pyridazin-3-ylamine), GSK1120212, VX745 and SU5402 (2-[(1,2-Dihydro-2-oxo-3H-indol-3-ylidene)methyl]-4-methyl-1H-pyrrole-3-propanoic acid).

In one embodiment, the modulators of TGFβ signaling may include but are not limited to activators such as Activin A, TGFβ1, TGFβ2, TGFβ3, IDE1 (1-[2-[(2-Carboxyphenyl)methylene]hydrazide]heptanoic acid), IDE2 (Heptanedioic acid-1-(2-cyclopentylidenehydrazide) or Nodal, or may include but are not limited to inhibitors such as A-83-01 (3-(6-Methyl-2-pyridinyl)-N-phenyl-4-(4-quinolinyl)-1H-pyrazole-1-carbothioamide), SB431542 (4-[4-(1,3-benzodioxol-5-yl)-5-pyridin-2-yl-1H-imidazol-2-yl]benzamide), SB-505124 (2-[4-(1,3-Benzodioxol-5-yl)-2-(1,1-dimethylethyl)-1H-imidazol-5-yl]-6-methyl-pyridine), Lefty1 and Lefty 2.

In one embodiment, the modulators of PKA and cAMP signaling may include but are not limited to activators such as 8-bromoCAMP, Forskolin and sp-CAMP.

In one embodiment, the modulators of Notch signaling may include but are not limited to gamma secretase inhibitors such as R04929097 (Propanediamide, N1-[(7S)-6,7-dihydro-6-oxo-5H-dibenz[b,d]azepin-7-yl]-2,2-dimethyl-N3-(2,2,3,3,3-pentafluoropropyl)-) and DAPT (N-[(3,5-Difluorophenyl)acetyl]-L-alanyl-2-phenyl]glycine-1,1-dimethylethyl ester).

In one embodiment, the modulators of PKG signaling may include but are not limited to inhibitors such as 1400 W dihydrochloride (N-[[3-(Aminomethyl)phenyl]methyl]-ethanimidamide dihydrochloride) and KT5823 ((9S,10R,12R)-2,3,9,10,11,12-Hexahydro-10-methoxy-2,9-dimethyl-1-oxo-9,12-epoxy-1H-diindolo[1,2,3-fg:3′,2′,1′-kl]pyrrolo[3,4-i][1,6]benzodiazocine-10-carboxylic acid, methyl ester). In one embodiment, the modulators of PKG signaling may include but are not limited to activators such as cyclic GMP and SNAP.

In one embodiment, the modulators of Oncostatin M/Gp130 signaling may include but are not limited to Oncostatin M (OSM), or other family members of the Oncostatin M or Gp130 signaling pathway.

In one embodiment, the one or more modulators of steroid hormone signaling may include but are not limited to progesterone or gluccocorticoid. In one embodiment, the modulators of glucocorticoid may include but are not limited to agonists such as dexamethasone (DEX), Cortisol, Dexamethasone-t-butylacetate, Hydrocortisone and GSK9027 (N-[4-[1-(4-Fluorophenyl)-1H-indazol-5-yl-3-(trifluoromethyl) phenyl]benzenesulfonamide).

In one embodiment, the modulators of Wnt signaling may include but are not limited to activators such as CHIR99201 (6-[[2-[[4-(2,4-dichlorophenyl)-5-(5-methyl-1H-imidazol-2-yl)-2pyrimidinyl]amino]ethyl]amino]-3-pyridinecarbonitrile), A1070722 (1-(7-Methoxyquinolin-4-yl)-3-[6-(trifluoromethyl)pyridin-2-yl]urea), Wnt3a, acetoxime, BIOacetoxime, BIO (6-bromo-3-[(3E)-1,3-dihydro-3-(hydroxyimino)-2H-indol-2-ylidene]-1,3-dihydro-(3Z)-2H-indol-2-one), members of the R-spondin family, or members of the Wnt signaling family. In one embodiment, the modulators of Wnt signaling may include but are not limited to inhibitors such as C59 (2-(4-(2-methylpyridin-4-yl)phenyl)-N-(4-(pyridin-3-yl)phenyl)acetamide), IWP2 (N-(6-Methyl-2-benzothiazolyl)-2-[(3,4,6,7-tetrahydro-4-oxo-3-phenylthieno[3,2-d]pyrimidin-2-yl)thio]-acetamide), Dkk1, XAV939 (3,5,7,8-Tetrahydro-2-[4-(trifluoromethyl)phenyl]-4H-thiopyrano[4,3-d]pyrimidin-4-one), IWR1 (4-(1,3,3a,4,7,7a-Hexahydro-1,3-dioxo-4,7-methano-2H-isoindol-2-yl)-N-8-quinolinyl-Benzamide) FH-535=(2,5-Dichloro-N-(2-methyl-4-nitrophenyl)benzenesulfonamide.

In one embodiment, the one or more modulators of vitamin D signaling may include but are not limited to activators such as cholecalciferol or Vitamin D3.

In one embodiment, the one or more activators of ascorbic acid signaling may include but are not limited to activators such as L-ascorbic acid 2-phosphate (AA2P) or L-ascorbic acid or 2-O-(beta-D-Glucopyranosyl)ascorbic acid.

In one embodiment, the one or more activators of insulin signaling may include but are not limited to activators such as Insulin or Insulin-like Growth Factor-1 (IGF1).

In one embodiment, the phospholipid precursors may include but are not limited to ethanolamine, choline chloride, inositol, serine or glycerol.

In one embodiment, the modulator of growth factor signaling may include but are not limited to family member proteins of any one of the signaling pathways of Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Tumor necrosis factor-alpha(TNF-α), Vascular endothelial growth factor (VEGF), placental growth factor (PlGF), Foetal Bovine Somatotrophin (FBS), IL-1, IL-2, IL-3, IL-4, IL-5, IL-6 or IL-7.

The differentiation factors disclosed herein may be used in an amount from about 0.01 ng/ml to about 200 jag/ml, or from about 0.5 ng/ml to about 150 jag/ml, or about 1 ng/ml 5 to about 100 jag/ml, or about 10 ng/ml to about 100 jag/ml, or about 15 ng/ml to about 50 jag/ml.

The differentiation factors disclosed herein may be used in an amount that ranges from about 0.1 nM to IM, or from about 0.1 nM to about 200 mM, or from about 0.5 nM to about 150 mM, or about 0.5 nM to about 100 mM, or about 1 nM to about 100 mM.

### Differentiating Cells of Definitive Endoderm (DE)

The present invention provides methods of differentiating cells of the definitive endoderm (DE) lineage into posterior foregut lineage (PFG).

Accordingly, in one embodiment, the present invention provides methods of differentiating stem cells of the definitive endoderm (DE) lineage into posterior foregut lineage and may comprise contacting said stem cells with: one or more retinoic acid activators; and/or one or more inhibitors of TGFβ signaling.

In one embodiment, the one or more retinoic acid activators may be in an amount of about 1 nM to 100 mM. In one embodiment, the one or more retinoic acid activators may comprise All-trans retinoic acid (ATRA) in an amount of about 2 μM and/or TTNPB in an amount of about 500 nM.

In one embodiment, the one or more inhibitors of TGFβ signaling may be in an amount of about 1 nM to 100 mM. In one embodiment, the one or more inhibitors of TGFβ signaling may comprise A83-01 in an amount of about 1 μM and/or SB431542 in an amount of about 10 μM and/or SB505124 in an amount of about 1-2 μM.

In one embodiment, the method may further comprise contacting said cells with one or more activators of BMP signaling. In one embodiment, the one or more activators of BMP signaling may be in an amount of about 1 ng/ml to 100 mg/ml. In one embodiment, the one or more activators of BMP signaling may comprise BMP4 in an amount of about 30 ng/ml and/or BMP2 in an amount of about 30 ng/ml and/or BMP7 in an amount of about 30 ng/ml.

In one embodiment, the method may further comprise contacting said stem cells with one or more activators of FGF signaling. In one embodiment, the one or more activators of FGF signaling may be in an amount of about 1 ng/ml to 100 mg/ml. In one embodiment, the one or more activators of FGF signaling may comprise FGF2 in an amount of about 10 ng/ml.

In one embodiment, the cells of the posterior foregut lineage may comprise elevated gene expression of posterior foregut lineage markers and decreased expression of dorsal foregut markers relative to undifferentiated cells. In one embodiment, the dorsal foregut marker comprises Mnx1.

In one embodiment, the differentiation of the cells of the definitive endoderm (DE) lineage into cells of the posterior foregut lineage may be completed from about 12 to 84 hours, 12 to 72 hours, 18 to 72 hours, 18 to 66 hours, 18 to 60 hours or 24 to 60 hours.

In one embodiment, the duration of the method may be about 1 to 84 hours.

### Differentiating Cells of the Posterior Foregut Lineage

The present invention provides methods of differentiating cells of the posterior foregut lineage into liver bud progenitors and may comprise contacting said cells of the posterior foregut lineage with one or more activators of TGFβ signaling, one or more modulators of Wnt signaling; and/or one or more activators of cyclic AMP/PKA signaling.

In one embodiment, the one or more activators of TGFβ signaling may be in an amount of about 1 ng/ml to 100 g/ml. In one embodiment, the one or more activators of TGFβ signaling may comprise Activin A in an amount of about 10 ng/ml and/or Nodal in an amount of about 10 ng/ml.

In one embodiment, the one or more modulators of Wnt signaling may be in an amount of about 1 nM to 1M. In one embodiment, the one or more modulators of Wnt signaling may comprise an inhibitor of Wnt signaling and an activator of Wnt signaling. In one embodiment, the activator of Wnt signaling may be contacted with the cells after the inhibitor of Wnt signaling has been contacted with the cells. In one embodiment, the inhibitor of Wnt signaling may be contacted with the cells of the posterior foregut lineage for duration of about 1 to 72 hours, and subsequently the activator of Wnt signaling may be contacted with the cells of the posterior foregut lineage for duration of about 24 to 48 hours. In one embodiment, the inhibitor of Wnt signaling may be C59 in an amount of about 1 μM and/or XAV in an amount of about 1 μM. In one embodiment, the activator of Wnt signaling may be CHIR in an amount of about 1 μM.

In one embodiment, the one or more activators of cyclic AMP/PKA signaling may be in an amount of about 1 nM to 1M. In one embodiment, the one or more activators of cyclic AMP/PKA signaling may comprise 8-bromoCAMP in an amount of about 1 mM and/or forskolin in an amount of about 10 μM.

In one embodiment, the method may further comprise contacting said cells of the posterior foregut lineage with one or more activators of BMP signaling. In one embodiment, the one or more activators of BMP signaling may be in an amount of about 1 ng/ml-100 g/ml. In one embodiment, the one or more activators of BMP signaling may comprise BMP4, BMP2, BMP7 or a combination thereof. In one embodiment, the one or more activators of BMP signaling may comprise BMP4 in an amount of about 30 ng/ml and/or BMP2 in an amount of about 30 ng/ml and/or BMP7 in an amount of about 30 ng/ml.

In one embodiment, the liver bud progenitors may comprise elevated gene expression of liver bud progenitor markers and decreased expression of pancreatic progenitor markers relative to undifferentiated cells.

In one embodiment, the liver bud progenitors may comprise elevated gene expression of markers comprising AFP, TBX3, HNF4A CEBPA, PROX1, HNF6, HNF1B, HNF1A or a combination thereof, relative to undifferentiated cells. In one embodiment, the liver bud progenitor markers may comprise Pdx1.

In one embodiment, the differentiation of cells of the posterior foregut lineage into liver bud progenitors may be completed from about 12 to 84 hours, 12 to 72 hours, 18 to 72 hours, 18 to 66 hours, 18 to 60 hours or 24 to 60 hours.

In one embodiment, the duration of the method may be for about 1 to 120 hours.

In one embodiment, the cells of the posterior foregut lineage may be obtained from the method of differentiating cells of the definitive endoderm (DE) lineage into posterior foregut lineage (PFG), as described herein.

### Maintaining Liver Bud Progenitors in a Self-Renewal State

The present invention provides methods of maintaining liver bud progenitors in a self-renewal state that may comprise contacting said liver bud progenitors with: one or more activators of FGF signaling, and/or one or more activators of Wnt signaling.

In one embodiment, the one or more activators of FGF signaling may comprise FGF2, FGF10, other family members of the FGF signaling pathway or a combination thereof.

In one embodiment, the one or more activators of Wnt signaling may comprise CHIR99201, Wnt3a, members of the R-spondin family, members of the Wnt signaling family, acetoxime, BIO or a combination thereof.

In one embodiment, the liver bud progenitors may be contacted with FGF and CHIR99201. In one embodiment, the liver bud progenitors are contacted with about 1 ng/mL to 1 mg/mL FGF and/or about 1 nM to 100 mM CHIR99201. In one embodiment, the liver bud progenitors may be contacted with about 20 ng/mL FGF and/or about 2 M CHIR99201.

In one embodiment, the method may further comprise contacting said liver bud progenitors with one or more activators of BMP signaling. In one embodiment, the one or more activators of BMP signaling may comprise BMP4, BMP2, BMP7 or a combination thereof.

In one embodiment, the method may further comprise contacting said liver bud progenitors with one or more epidermal growth factors.

In one embodiment, the method may further comprise contacting said liver bud progenitors with one or more inhibitors of Notch signaling. In one embodiment, the one or more inhibitors of Notch may comprise DAPT, R04929097 or a combination thereof.

In one embodiment, the method may further comprise contacting the liver bud progenitors with BMP4, EGF, and DAPT. In one embodiment, the liver bud progenitors may be contacted with about 1 ng/mL to 1 mg/mL BMP4 and/or about 1 ng/mL to 1 mg/mL EGF, and/or about 1 nM to 100 mM DAPT. In one embodiment, the liver bud progenitors may be contacted with about 10 ng/mL BMP4, and/or about 10 ng/mL EGF, and/or about 10 μM DAPT.

In one embodiment, the liver bud progenitors may comprise elevated gene expression of markers comprising AFP, TBX3, HNF4a, CEBPα or a combination thereof, relative to differentiated cells.

In one embodiment, the liver bud progenitors may be obtained from the method of differentiating cells of the posterior foregut lineage into liver bud progenitors, as described herein.

### Differentiating Liver Bud Progenitors

The present invention provides methods of differentiating liver bud progenitors into hepatic progenitors that may comprise contacting said liver bud progenitors with: one or more inhibitors of TGFβ signaling; one or more inhibitors of FGF signaling; and/or one or more inhibitors of Notch signaling.

In one embodiment, the method may further comprise contacting the cells with: one or more inhibitors of Notch signaling; one or more activators of ascorbic acid signaling, one or more activators of cyclic AMP/PKA signaling; and/or one or more activators of insulin signaling or a combination thereof.

In one embodiment, the method may further comprises contacting said liver bud progenitors with one or more differentiation factors that may be selected from the group comprising of: activators of ascorbic acid signaling; activators of glucocorticoid signaling; activators of cyclic AMP/PKA signaling; activators of insulin signaling; activators of oncostatin M signaling; an amino acid mixture, and/or activators of L-glutathione signaling.

In one embodiment, the method may further comprises contacting the cells with one or more differentiation factors that may be selected from the group comprising of: activators of BMP signaling; inhibitors of PKG signaling; activators of glucocorticoid signaling; phospholipid precursors; activators of oncostatin M signaling; an amino acid mixture, and/or activators of L-glutathione signaling or a combination thereof.

In one embodiment, the one or more inhibitors of TGFβ signaling may be in an amount of about 1 nM-100 mM. In one embodiment, the one or more inhibitors of TGFβ signaling may comprise A83-01 in an amount of about 1 μM and/or SB431542 in an amount of about 10 μM and/or SB505124 in an amount of about 1-2 μM.

In one embodiment, the one or more inhibitors of FGF signaling may be in an amount of about 1 nM-100 mM. In one embodiment, the one or more inhibitors of FGF signaling may comprise GSK1120212 in an amount of about 250 nM and/or PD173074 in an amount of about 100 nM and/or VX745 in an amount of about 250 nM.

In one embodiment, the one or more inhibitors of Notch signaling may be in an amount of about 1 nM-100 mM. In one embodiment, the one or more inhibitors of Notch signaling may comprise R04929097 in an amount of about 2 μM and/or DAPT in an amount of about 10 μM.

In one embodiment, the one or more activators of ascorbic acid signaling may be in an amount of about 1 ng/ml-100 mg/ml. In one embodiment, the one or more activators of ascorbic acid signaling may comprise AA2P in an amount of about 200 μg/ml, and/or L-ascorbic acid in an amount of about 200 μg/ml and or 2-O-(beta-D-Glucopyranosyl)ascorbic acid at about 200 μg/ml.

In one amount, the one or more activators of cyclic AMP/PKA signaling may be in an amount of about 1 nM-1M. In one embodiment, the one or more activators of cyclic AMP/PKA signaling may comprise 8-bromoCAMP in an amount of about 1 mM and/or forskolin in an amount of about 10 μM.

In one embodiment, the one or more activators of insulin signaling may be in an amount of about 1 ng/ml-100 mg/ml. In one embodiment, the one or more activators of insulin signaling may comprise insulin in an amount of about 10 μg/ml.

In one embodiment, the activators of glucocorticoid signaling may be in an amount of about 1 nM-100 mM. In one embodiment, the one or more activators of glucocorticoid signaling may comprise DEX in an amount of about 10 μM and/or GSK9097 in an amount of about 10 μM.

In one embodiment, the activators of oncostatin M signaling may be in an amount of about 1 ng/ml-100 mg/ml. In one embodiment, the one or more activators of oncostatin M signaling may comprise OSM, other family members of the oncostatin M or Gp130 signaling pathway of about 10 ng/ml, or a combination thereof.

In one embodiment, the one or more activators of BMP signaling may be in an amount of about 1 ng/ml-100 mg/ml. In one embodiment, the one or more activators of BMP signaling may comprise BMP4, BMP2, BMP7 or a combination thereof. In one embodiment, the one or more activators of BMP signaling may comprise BMP4 in an amount of about 30 ng/ml and/or BMP2 in an amount of about 30 ng/ml and/or BMP7 in an amount of about 30 ng/ml.

In one embodiment, the phospholipid precursors may be in an amount of 0.001%-5% or 1 ng/ml-100 mg/ml. In one embodiment, the phospholipid precursors may comprise ethanolamine in an amount of 0.02% and/or choline chloride in an amount of about 50 μg/ml. As will be appreciated in the art, the % amounts refer to an amount per 100 ml. For example, 1% refers to 1 ml/100 ml and 0.02% refers to 20 ul/100 ml.

In one embodiment, the inhibitors of PKG signaling may be in an amount of about 1 nM-100 mM. In one embodiment, the inhibitors of PKG signaling may comprise 1400 W dihydrochloride in an amount of about 2 μM and/or KT5823 in an amount of about 2 μM and/or (S)-Methylisothiourea sulfate at about 1 μM.

In one embodiment, the activators of L-glutathione signaling may be in an amount of about 1 ng/ml-100 mg/ml. In another embodiment, the activators of L-glutathione signaling may comprise L-glutathione in an amount of about 100 g/ml.

In one embodiment, the amino acid mixture may be in a concentration of about 1 ng/ml or greater, and may comprise Glycine, L-Alanine, L-Arginine, L-Asparagine, L-Aspartic acid, L-Cysteine hydrochloride, L-Glutamic Acid, L-Glutamine, L-Histidine hydrochloride, L-Isoleucine, L-Leucine, L-Lysine hydrochloride, L-Methionine, L-Phenylalanine, L-Proline, L-Serine, L-Threonine, L-Tryptophan, L-Valine and L-Tyrosine. In one embodiment, the amino acid mixture may comprise Glycine in an amount of about 187.5 mg/L, L-Alanine in an amount of about 169.5 mg/L, L-Arginine hydrochloride in an amount of about 1475 mg/L, L-Asparagine in an amount of about 200.05 mg/L, L-Aspartic acid in an amount of about 216.5 mg/L, L-Cysteine hydrochloride in an amount of about 632.6 mg/L, L-Glutamic Acid in an amount of about 448.5 mg/L, L-Glutamine in an amount of about 2 mM, L-Histidine hydrochloride in an amount of about 315 mg/L, L-Isoleucine in an amount of about 545 mg/L, L-Leucine in an amount of about 590.5 mg/L, L-Lysine hydrochloride in an amount of about 912.5 mg/L, L-Methionine in an amount of about 172.5 mg/L, L-Phenylalanine in an amount of about 355 mg/L, L-Proline in an amount of about 372.5 mg/L, L-Serine in an amount of about 262.5 mg/L, L-Threonine in an amount of about 534.5 mg/L, L-Tryptophan in an amount of about 90.02 mg/L, L-Valine in an amount of about 525.35 mg/L, L-Tyrosine in an amount of about 559.05 mg/L

In one embodiment, the hepatic progenitors may comprise elevated gene expression of hepatic markers and decreased expression of biliary markers relative to undifferentiated cells. In one embodiment, the hepatic progenitors may comprise elevated gene expression of markers comprises ALBUMIN, c-MET, HNF4A, CEBPα or a combination thereof, relative to undifferentiated cells. In one embodiment, the hepatic progenitors comprise decreased gene expression of marker SOX9 relative to undifferentiated cells.

In one embodiment, the differentiation of liver bud progenitors into hepatic progenitors may be completed from about 12 to 84 hours, 12 to 72 hours, 18 to 72 hours, 18 to 66 hours, 18 to 60 hours or 24 to 60 hours.

In one embodiment, the duration of step a) may be for about 1 to 108 hours. In another embodiment, the duration of step b) may be for at least 48 hours. In one embodiment, the duration of the method may be at least 156 hours.

In one embodiment, the liver bud progenitors may be obtained from the method of differentiating cells of the posterior foregut lineage into liver bud progenitors, as described herein.

Differentiating Hepatic Progenitors into Perivenous Hepatocyte-Like Cells

The present invention provides methods of differentiating hepatic progenitors into perivenous hepatocyte-like cells that may comprise contacting said hepatic progenitors with: one or more inhibitors of Notch signaling, one or more activators of glucocorticoid signaling, one or more activators of insulin signaling; one or more activators of ascorbic acid signaling, and/or one or more activators of TGFβ signaling.

In one embodiment, the one or more inhibitors of Notch signaling may be in amount of about 1 nM-100 mM. In one embodiment, the one or more inhibitors of Notch signaling may comprise R04929097 in an amount of about 2 μM and/or DAPT in an amount of about 10 μM.

In one embodiment, the one or more activators of glucocorticoid signaling may be in an amount of about 1 nM-100 mM. In one embodiment, the one or more activators of glucocorticoid signaling may comprise DEX in an amount of about 10 μM and/or GSK9097 in an amount of about 10 μM.

In one embodiment, the one or more activators of insulin signaling may be in an amount of about 1 ng/ml-100 mg/ml. In one embodiment, the one or more activators of insulin signaling may comprise insulin in an amount of about 10 μg/ml.

In one embodiment, the one or more activators of ascorbic acid signaling may be in an amount of about 1 ng/ml-100 mg/ml. In one embodiment, the one or more activators of ascorbic acid signaling may comprise AA2P in an amount of about 200 μg/ml, and/or L-ascorbic acid in an amount of about 200 μg/ml.

In one embodiment, the one or more activators of TGFβ signaling may be in amount of about 1 ng/ml-100 mg/ml. In one embodiment, the one or more activators of TGFβ signaling may comprise Activin in an amount of about 100 ng/ml and/or Nodal in an amount of about 100 ng/ml.

In one embodiment, the method may further comprise contacting the hepatic progenitors with one or more activators of retinoic acid signaling. In one embodiment, the one or more activators of retinoic acid signaling may be in an amount of about 1 nM-100 mM. In one embodiment, the one or more activators of retinoic acid signaling may comprise 9-cisRA in an amount of about 1 μM.

In one embodiment, the method may further comprise contacting said hepatic progenitors with one or more activators of progesterone signaling. In one embodiment, the one or more activators of progesterone signaling may be in an amount of 1 nM to 1 mM. In one embodiment, the one or more activators of progesterone signaling may comprise progesterone in an amount of about 1 μM.

In one embodiment, the method may further comprise contacting said hepatic progenitors with one or more activators of vitamin D signaling. In one embodiment, the one or more activators of vitamin D signaling may be in an amount of about 1 ng/mL to 1 mg/mL. In one embodiment, the one or more activators of vitamin D signaling may comprise cholecalciferol in an amount of about 500 nM, and/or Vitamin D3 in an amount of about 500 nM.

In one embodiment, the method may further comprise contacting said hepatic progenitors with one or more activators of PKG signaling. In one embodiment, the one or more activators of PKG signaling may comprise cGMP or S-nitrosoacetyl penicillamine (SNAP).

In one embodiment, the perivenous hepatocyte-like cells may comprise elevated gene expression of perivenous hepatocytes markers and decreased expression of periportal markers relative to undifferentiated cells. In one embodiment, the perivenous hepatocyte markers may comprise GS, CYP3A4, AAT, AXIN2 or a combination thereof.

In one embodiment, the differentiation of hepatic progenitors into perivenous hepatocyte-like cells may be completed from about 12 to 84 hours, 12 to 72 hours, 18 to 72 hours, 18 to 66 hours, 18 to 60 hours or 24 to 60 hours. In one embodiment, the differentiation may be completed within at least 1 hour.

In one embodiment, the duration of the method may be about 1 to 168 hours.

In one embodiment, the hepatic progenitors may be obtained from the method of differentiating liver bud progenitors into hepatic progenitors, as described herein.

### Maintaining Hepatocytes or Hepatocyte-Like Cells in a Perivenous State

The present invention provides methods of maintaining hepatocytes or hepatocyte-like cells in a perivenous state that may comprise contacting said cells with one or more inhibitors of Notch signaling and/or one or more activators of Wnt signaling such that the expression of perivenous cell markers may be maintained at high levels during in vitro cell culture.

In one embodiment, the method may further comprise contacting said cells with: one or more inhibitors of TGFβ; and/or one or more activators of estrogen signaling such as estradiol.

In one embodiment, the cells may be obtained from the method of differentiating hepatic progenitors into perivenous hepatocytes, as described herein. In one embodiment, the cells may be obtained from human livers.

Differentiating Hepatic Progenitors into Hepatocyte-Like Cells

The present invention provides methods of differentiating hepatic progenitors into hepatocyte-like cells that may comprise contacting said hepatic progenitors with: one or more activators of PKA signaling; one or more activators of glucocorticoid signaling; one or more activators of insulin signaling; one or more activators of ascorbic acid signaling, and one or more inhibitors of Notch signaling.

In one embodiment, the one or more activators of cyclic AMP/PKA signaling may be in an amount of about 1 nM-1M. In one embodiment, the one or more activators of PKA signaling may comprise 8-bromoCAMP in an amount of about 1 mM, and/or forskolin in an amount of about 10 μM and/or 16,16-dimethyl-prostaglandin E2 (16,16-dmPGE2) in an amount of 10 μM.

In one embodiment, the one or more activators of glucocorticoid signaling may be in an amount of about 1 nM-100 mM. In one embodiment, the one or more activators of glucocorticoid signaling may comprise DEX in an amount of about 10 μM and/or GSK9097 in an amount of about 10 μM.

In one embodiment, the one or more activators of insulin signaling may be in an amount of about 1 ng/ml-100 mg/ml. In one embodiment, the one or more activators of insulin signaling may comprise insulin in an amount of about 10 μg/ml.

In one embodiment, the one or more activators of ascorbic acid signaling may be in an amount of about 1 ng/ml-100 mg/ml. In one embodiment, the one or more activators of ascorbic acid signaling may comprise AA2P in an amount of about 200 μg/ml, and/or L-ascorbic acid in an amount of about 200 μg/ml.

In one embodiment, the one or more inhibitors of Notch signaling may be in an amount of about 1 nM-100 mM. In one embodiment, the one or more inhibitors of Notch signaling may comprise R04929097 in an amount of about 2 μM and/or DAPT in an amount of about 10 μM.

In one embodiment, the method may further comprise contacting said hepatic progenitors with one or more inhibitors of Wnt signaling. In one embodiment, the one or more inhibitors of Wnt signaling may be in an amount of about 1 nM-1M. In one embodiment, the one or more inhibitors of Wnt signaling may comprise C59 in an amount of about 1 μM, and/or XAV in an amount of about 1 μM.

In one embodiment, the method may further comprise contacting said hepatic progenitors with one or more inhibitors of PKG signaling. In one embodiment, the one or more inhibitors of PKG signaling may be in an amount of about 1 nM-100 mM. In one embodiment, the one or more inhibitors of PKG signaling may comprise 1400 W dihydrochloride in an amount of about 2 μM, and/or KT5823 in an amount of about 2 μM or (S)-Methylisothiourea sulfate at about 1 μM.

In one embodiment, the hepatocyte-like cells may be periportal hepatocytes.

In one embodiment, the hepatocyte-like cells may comprise elevated gene expression of hepatocyte-like cell markers and decreased expression of perivenous markers relative to undifferentiated cells. In one embodiment, the hepatocyte-like cell markers may comprise CPS1, TAT, Albumin, APC, ARG1 or a combination thereof.

In one embodiment, the differentiation of hepatic progenitors into hepatocyte-like cells may be completed from about 12 to 84 hours, 12 to 72 hours, 18 to 72 hours, 18 to 66 hours, 18 to 60 hours or 24 to 60 hours. In one embodiment, the differentiation may be completed within at least 1 hour.

In one embodiment, the duration of the method may be for about 1 to 168 hours.

In one embodiment, the hepatic progenitors may be obtained from the method of differentiating liver bud progenitors into hepatic progenitors, as described herein.

### Maintaining Hepatocytes in a Periportal State

The present invention provides methods of maintaining periportal heaptocytes in a self-renewal state that may comprise contacting said periportal heaptocytes with one or more activators of cyclic AMP/PKA signaling.

In one embodiment, the periportal hepatocytes may be obtained from the method of differentiating hepatic progenitors into hepatocyte-like cells, as described herein. In one embodiment, the cells may be obtained from human livers.

### Cell Culture Media and Kits

The methods and cells described herein may be contacted with the one or more differentiating factors in a culture medium supplemented with other factors or otherwise processed to adapt it for propagating, maintaining or differentiation of the cells lineages. To maintain stem cell pluripotency, for example, the stem cells and cell lineages disclosed herein may be cultured in conditioned medium, such as mEF-CM, or fresh serum-free medium alone, mTesR, or other hPSC maintenance media that are known in the art or xeno-free media such as Essential 8. To differentiate stem cells the stem cells and cell lineages disclosed herein may be cultured in a feeder free medium or medium comprising a feeder layer, whereby the culture mediums may be CDM2, CDM KOSR or IMDM/F12. CDM2 comprises of chemically defined as containing Iscove's Modified Dulbecco's Media (IMDM), Ham's F12 nutrient mixture (F12), transferrin, insulin, concentrated lipids, or polyvinyl alcohol (PVA). CDM KOSR comprises of 10% KOSR, IMDM, F12 concentrated lipids, or PVA. IMDM/F12 comprises of IMDM and F12. IMDM/F12 media has been used between days 7-18 as the certain components in KOSR appears to promote lipid formation. Alternatively, for differentiation a basal media may be used derived from minimal basal media that contain the basic ingredients for cell survival and growth known in the art, and that do not contain added growth factors/chemicals that confound differentiation. Such factors may be supplied in the form of a kit to be added to, or be used in the preparation of a culture medium for use in propagating, maintaining or differentiation of the cell lineages described herein.

In one embodiment, the culturing of the cells may be in formats including but not limited to a monolayer culture, an aggregate culture, or a suspension culture. As will be appreciated in the art, in a monolayer culture, the cells may adhere to a support such as a plastic support or a matrix while in a suspension culture, whereby cells may not adhere to any surface. As will be appreciated in the art, in an aggregate culture, the cells may be grown in contact with other cells as “balls” or “clumps” or “aggregates” of cells.

In one embodiment, the culture medium may be a conditioned medium obtained from a feeder layer. It is contemplated that the feeder layer comprises fibroblasts, and in one embodiment, comprises embryonic fibroblasts.

An alternative culture system employs serum-free medium supplemented with growth factors capable of promoting the proliferation of stem cells. For example, a feeder-free, serum-free culture system in which stem cells are maintained in unconditioned serum replacement (SR) medium supplemented with different growth factors capable of triggering stem cell self-renewal.

In one embodiment, the culture medium may be a feeder-free culture medium that may not contain feeder cells or exogenously added conditioned medium taken from a culture of neither feeder cells nor exogenously added feeder cells in the culture. Of course, if the cells to be cultured are derived from a seed culture that contained feeder cells, the incidental co-isolation and subsequent introduction into another culture of some small proportion of those feeder cells along with the desired cells (e. g., undifferentiated primate stem cells) should not be deemed as an intentional introduction of feeder cells. In such an instance, the culture contains a de minimus number of feeder cells. By “de minimus”, it is meant that number of feeder cells that are carried over to the instant culture conditions from previous culture conditions where the differentiable cells may have been cultured on feeder cells. Similarly, feeder cells or feeder-like cells that develop from stem cells seeded into the culture shall not be deemed to have been purposely introduced into the culture. Alternatively, a feeder free culture medium may be employed that is chemically defined and may contain PVA, concentrated lipids, knockout serum replacement (KOSR), IMDM, F12.

In one embodiment, the present invention provides a kit for differentiating cells of the definitive endoderm (DE) lineage into posterior foregut lineage that may comprise one or more retinoic acid activators, and one or more inhibitors of TGFβ signaling.

In another embodiment, the present invention provides a kit for differentiating cells of the posterior foregut lineage into liver bud progenitors that may comprise one or more activators of TGFβ signaling; one or more modulators of Wnt signaling; and one or more activators of cyclic AMP/PKA signaling.

In another embodiment, the present invention provides a kit for differentiating cells of the posterior foregut lineage into liver bud progenitors that may comprise one or more activators of TGFβ signaling; one or more modulators of Wnt signaling; one or more activators of PKA signaling and one or more activators of BMP signaling.

In another embodiment, the present invention provides a kit for differentiating liver bud progenitors into hepatic progenitors that may comprise one or more inhibitors of TGF signaling; one or more inhibitors of FGF signaling; and one or more inhibitors of Notch signaling.

In another embodiment, the present invention provides a kit for differentiating liver bud progenitors into hepatic progenitors that may comprise one or more inhibitors of Notch signaling; activators of ascorbic acid signaling; one or more activators of cyclic AMP/PKA signaling; and one or more activators of insulin signaling.

In another embodiment, the present invention provides a kit for differentiating hepatic progenitors into perivenous hepatocyte-like cells that may comprise one or more inhibitors of Notch signaling; one or more activators of glucocorticoid signaling; one or more activators of insulin signaling; one or more activators of ascorbic acid signaling; and one or more activators of TGFβ signaling.

In another embodiment, the present invention provides a kit for differentiating hepatic progenitors into hepatocytes or hepatocyte-like cells that may comprise one or more activators of cyclic AMP/PKA signaling; one or more activators of glucocorticoid signaling; one or more activators of insulin signaling; one or more activators of ascorbic acid signaling; and one or more inhibitors of Notch signaling.

In another embodiment, the present invention provides a kit for maintaining liver bud progenitors in a self-renewal state that may comprise one or more of the following factors: one or more activators of FGF signaling and one or more activators of Wnt signaling.

In one embodiment, the kit for maintaining liver bud progenitors in a self-renewal state may further comprises one or more of the following factors: one or more activators of BMP signaling, one or more epidermal growth factors, one or more inhibitors of TGFβ signaling, and one or more inhibitors of Notch signaling.

In one embodiment, there is provided a kit for maintaining hepatocytes or hepatocyte-like cells in a perivenous state comprising one or more of the following factors: one or more inhibitors of Notch signaling or one or more activators of Wnt signaling. The kit may further comprise one or more of the following factors: one or more inhibitors of TGFb; and/or one or more activators of estrogen signaling; one or more inhibitors of cyclic AMP/PKA signaling.

In one embodiment, there is provided a kit for maintaining hepatocytes or hepatocyte-like cells in a periportal state comprising one or more activators of cyclic AMP/PKA signaling.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The disclosure has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### Cell Surface Markers

In one embodiment, there is provided a surface marker for isolating or selecting for LB cells selected from EGFR or CD99.

In one embodiment, there is provided a surface marker for isolating or selecting for MHG cells comprising CD325 (N-cadherin).

### Screening Methods

In one embodiment, there is provided a method of screening for a cellular response, the method comprising: a) contacting a population of cells generated according to the methods as described herein with a pharmacological agent; and b) evaluating the population of cells for a cellular response induced by the pharmacological agent.

In one embodiment, the screening may be in vitro screening and the contacting step may be performed in vitro. The screening may be performed in vivo and the contacting step may be performed by administering the pharmacological agent to a host animal that comprises the population of cells.

In one embodiment, there is provided a method of screening for a phenotype, the method comprising: a) administering to a host animal a population of cells generated according to the methods as described herein, wherein the cells of the population of cells comprise a genetic modification in at least one genetic locus; and b) evaluating the host animal for a detectable phenotype induced by the administered population of cells.

In some embodiments, the genetic modification in at least one genetic locus may result in the disruption or deletion of at least one gene. The population of cells may comprise liver cells and the detectable phenotype may comprise a survival enhancement.

In one embodiment, there is provided a method of treating a subject for a condition, the method comprising: a) administering the subject a therapeutically effective amount of cells generated according to the methods described herein in order to treat the subject for the condition.

The cells may be co-administered with at least one pro-survival or pro-engraftment factor.

The cells may comprise a genetic modification in at least one genetic locus.

In one embodiment there is provided use of a therapeutically effective amount of cells generated according to the methods as described herein in the manufacture of a medicament for treating a condition in a subject.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Materials and Methods

BCL2-Overexpressing hESCs

mTeSR1-grown H9 hESCs were transduced with an EF1A-BCL2-T2A-GFP lentivirus (C306). Successfully-transduced GFP+ hESCs were enriched through two rounds of FACS sorting to yield a >90% pure polyclonal BCL2-GFP+ hESC population. C306 H9 hESCs were subsequently used for liver differentiation.

### Tissue Culture

mTeSR1-grown hPSCs were cultured and differentiated as follows:
a) hPSCs cells were seeded at 1:12 as small clumps using Accutase (Millipore, Cat#SCR005) and Geltrex (Lifetech, Cat#A1413302).
b) Anterior Primitive Streak (day 1) was specified using Activin A (100 ng/ml), CHIR99021 (3 μM), PI-103 (50 nM) and Fgf2 (10 ng/ml) in serumless CDM2 basal medium for 24 hr.
c) Activin A (100 ng/ml) and DM3189 (250 nM) and PI-103 (50 nM) were added in the same basal medium for 24 hr to induce Definitive Endoderm (DE) (day 2).
d) DE was then patterned into PFG by using A83-01 (1 μM), RA (2 μM), BMP4 (30 ng/ml) and Fgf2 (10 ng/ml) in CDM KOSR basal media for another 24 hrs.
e) PFG was further differentiated to LB progenitors using Activin A (100 ng/ml), 8-bromo-CAMP (1 mM) and BMP4 (10 ng/ml) in CDM KOSR basal media for 3 days (day 4 to 6).
f) LB progenitors were treated with BMP4 (10 ng/ml), Oncostatin M (10 ng/ml), Dexamethasone (10M), DAPT, C59 (1 μM) or RO4919097 (2 μM), forskolin (10M), human recombinant insulin (10 μg/ml), ascorbic acid-2 phosphate (200 μg/ml), L-gluthathione (100 g/ml) and amino acid concentrate and in IMDM/F12 media for the next 6 days (day 7 to 12) to induce hepatic progenitors.
g) Hepatic progenitors were treated with Dexamethasone (10 μM), RO4929097 (2 μM) or DAPT (10 μM), Forskolin (10 μM), human recombinant insulin (10 μg/ml), ascorbic acid-2 phosphate (200 μg/ml) in IMDM/F12 media for 6 days (day 13 to 18) to generate hepatocyte-like cells.
   Intra-Splenic Transplantation into FRG^{-/-} Mice

FRG^{-/-} C57BL6 mice were purchased from Yecuris Corporation and maintained with 16 mg/L NTBC water. 1 day before surgery, the FRG−/− mice were retro-orbitally injected with adenovirus expressing uPA at 1.25*10̂9 pfu/25 gram. Approximately 24 hours later, the mice were intra-splenically transplanted with 1.5 million BCL2 OEhPSC-derived liver cells. 1 ml 0.9% Saline, painkillers (1.5 mg/kg Buproepinephrine) and antibiotics (10 mg/kg Enrofloxacin) were administered subcutaneously to the mice immediately after and 3 days post-operation. The mass of the mice was measured every 2 weeks.

During experimental cycling of Drug 2-(2-nitro-4-fluoromethylbenzoyl)-1,3-cyclohexanedione (NTBC), 8 mg/L NTBC was supplied for 3 days after 7 days of NTBC OFF period. The period of NTBC removal was increased progressively. For example, NTBC was removed for 8, 9, 10, 11, 12, 13, 14 days gradually over 2-4 months. Thereafter, the NTBC was removed completely. The same amount of NTBC was provided to each cage to standardize as much as possible the extent of injury. Care was taken as to whether each mouse received the full dose of NTBC.

### Gene Expression Profiling

RNA was extracted using Zymo RNA extraction kit. RT-quantitative PCR was used to quantify gene expression. Primer sequences were designed by NCBI Primer Tool and primer specificity and linearity was tested before usage for gene expression analyses.

### Immunostaining and Immunohistochemistry

TABLE 3
Antibodies

| | | | | Catalog |
|---|---|---|---|---|
| Antibody | Host | Dilution | Vendor | Number |
| Anti-ALBUMIN | Rabbit | IHC | Abcam | Ab2406 |
| | | IF | | |
| Anti-FAH | Rabbit | IHC | Abcam | Ab81087 |
| | | WB | | Ab83770 |
| Anti-HNF4A | Goat | IF | R&D | AF4605 |
| Anti-TBX3 | Goat | IF | R&D | AF4509 |
| Anti-AFP | Rabbit | IF | DakoCytomation | A000829 |
| Anti-CEBPA | Mouse | IF | Abcam | ab128482 |
| Anti-HNF6 | Sheep | IF | R&D | AF6277 |
| Anti-PROX1 | Goat | IF | R&D | AF2727 |
| Anti-HNF1B | Goat | IF | Santa Cruz | SC-7411 |

### Flow Cytometry

Legend screen was conducted as per manufacturers' instructions. Flow cytometry analyses was conducted using LSRII and FlowJo.

### EXPERIMENTAL RESULTS

I. Definitive Endoderm (DE) Differentiation into Posterior Foregut (PFG)
Retinoic Acid, BMP, TGFβ and FGF Signaling Defines Hepatopancreatic Domain and Fosters Endoderm with Hepatic-Pancreatic Competence.

During liver development, populations of definitive endoderm cells are first patterned along the anterior-posterior axis to form either the foregut or mid/hindgut. In the present invention the effects of signaling inhibition on the developmental competence of the foregut cells to give rise to the liver bud (LB) cells was examined. Competence is defined as the capacity of one stem cell or progenitor cell to differentiate into its daughter cell type. For example, the posterior foregut is competent when it could give rise to its daughter cell types such as the pancreatic endoderm and LB cells.

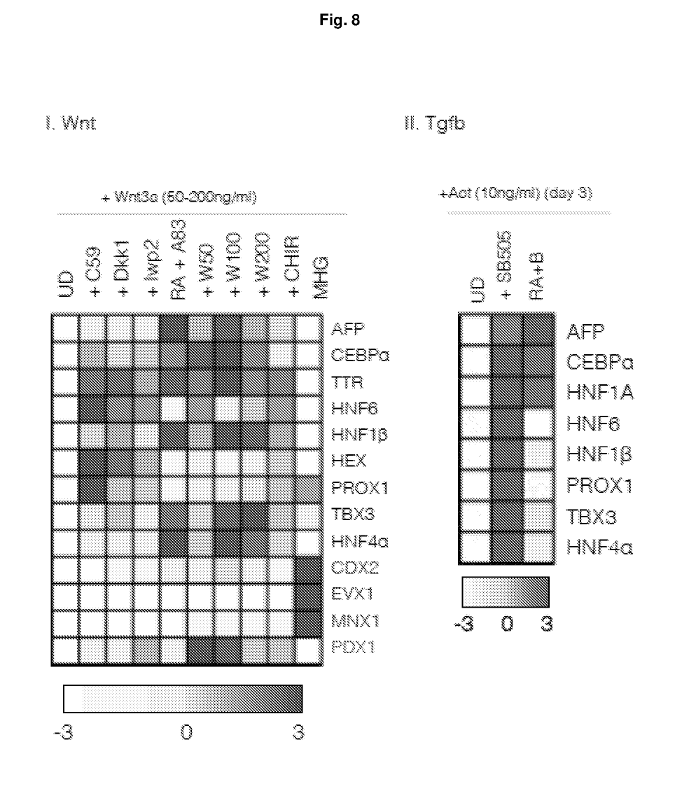

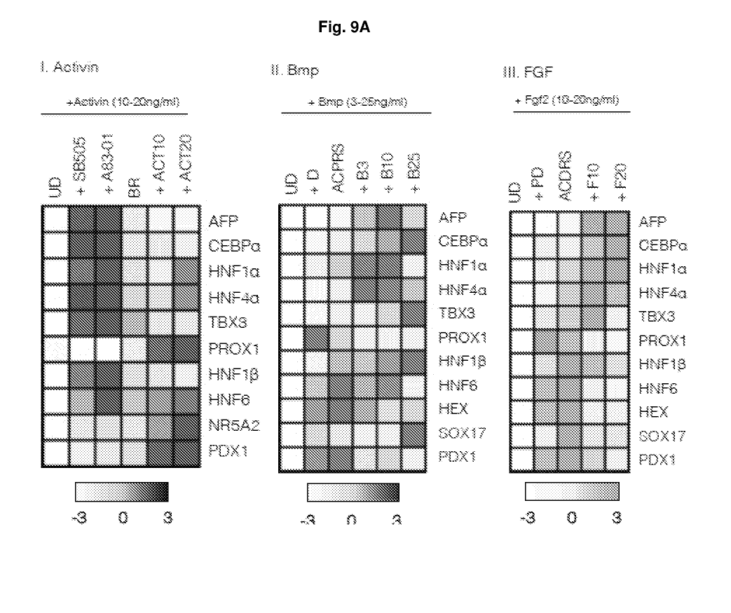

In particular, it is important to note that in vivo, foregut (FG) and midgut/hindgut (MHG) are anterior-posteriorly (AP) patterned in a mutually exclusive fashion. For example, Wnt inhibition has been shown to promote expression of foregut marker Hex and repress midgut/hindgut specification, whilst Wnt activation conversely promoted midgut/hindgut cell fate and inhibited foregut marker expression in zebrafish embryos. However, in the present invention it is shown that while Wnt inhibition by DKK1, C59 and IWP2 (24 h) during foregut specification reduces expression of MHG markers CDX2 and EVX1, it also severely reduces the expression of downstream LB gene expression (AFP, CEBPA, TBX3, HNF1B) (FIG. 8A). On the other hand, HNF6, PROX1 and HEX (expressed in early pancreatic and liver cells) expression were upregulated upon Wnt inhibition (FIG. 8A).

In this regard, it was found that a combination of TGFβ inhibition and treatment with BMP, FGF and RA generated posterior foregut (PFG)-like cells that are competent to generate hepatic cells.

### Retinoic Acid (RA)

Retinoic acid (RA) was found to endow endoderm with hepatic-pancreatic competence.

In xenopus, RA at the end of gastrulation affects both specification of liver and the pancreas. In zebrafish embryos, RA signaling is also known to endogenously pattern gut endoderm along the anterior-posterior axis. For example, zebrafish embryos deficient in RA also lack expression of Pdx1 and consequently fail to induce pancreas development.

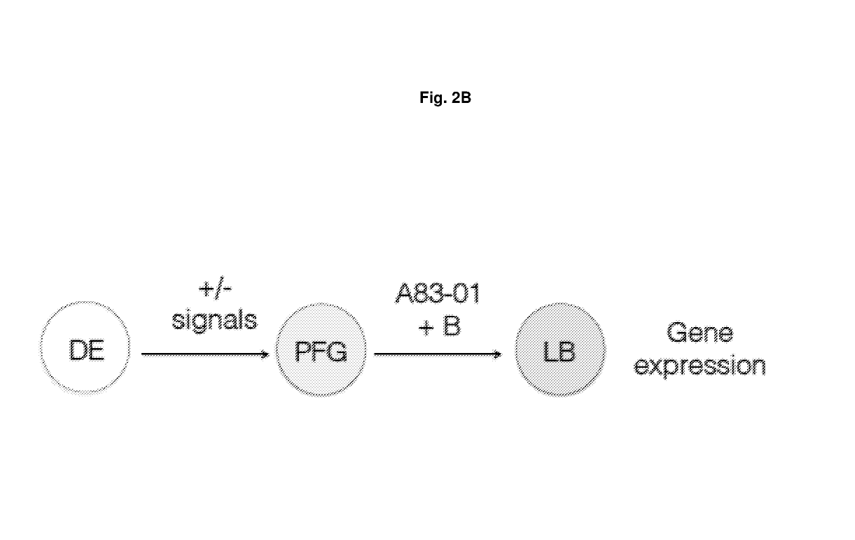

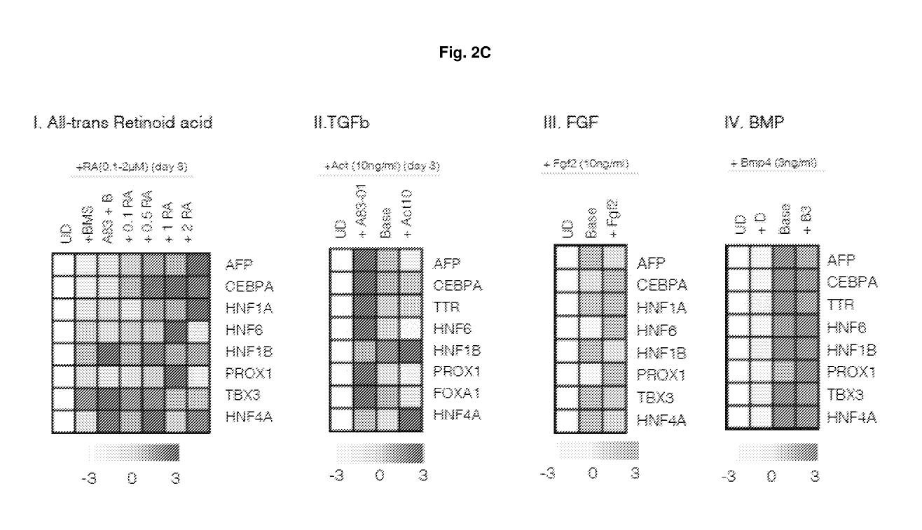

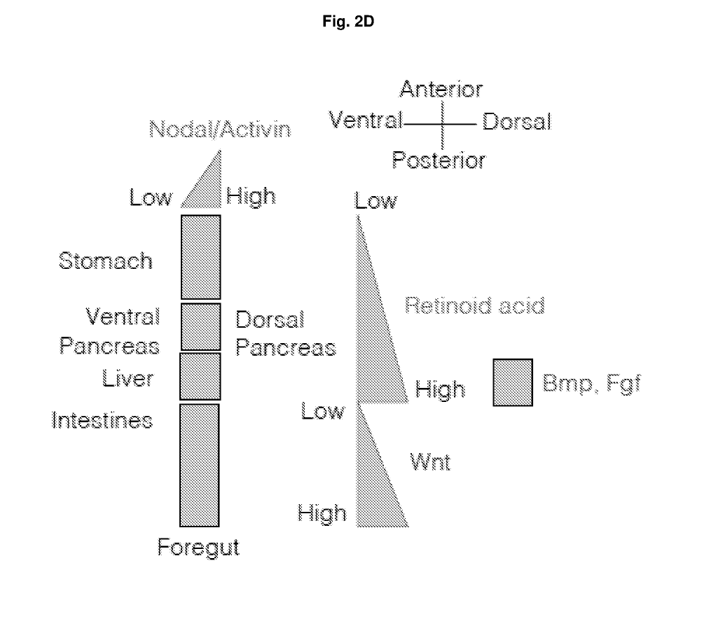

By treating day 2 hPSC-derived DE with increasing doses of RA, it was found that the addition of RA enhances hepatic competence as shown by the subsequently increased expression of AFP, HNF4A, and CEBPA in the LB cells (FIG. 2).

### BMP Signaling

It was found that BMP inhibition (24 h) after DE specification reduces competence of cells to differentiate into LB cells and reduces subsequent liver gene expression of AFP, CEBPA, TBX3, TTR, HNF4A, PROX1, HNF6, HNF1B. Conversely, BMP4 treatment during foregut specification, leads to mildly enhanced competence to generate LB cells (FIG. 2).

### TGFβ Signaling

TGFβ inhibition treatment on DE cells during foregut specification stage results in later increase in liver gene expression (AFP, CEBPA, TBX3, HNF6, PROX1, FOXA1, TTR) and favors subsequent LB differentiation (FIG. 2).

### FGF Signaling

In this regard, by providing FGF2 to DE cells during foregut specification, a subsequent increase in liver bud gene expression of CEBPA, HNF1A, HNF6, PROX1 was observed (FIG. 2).

II. Posterior Foregut (PFG) Differentiation into Liver Bud (LB)
PKA, BMP, WNT, FGF and TGFβ Promotes while RA Inhibits Liver Bud Specification

The signaling pathways, including Retinoic, Nodal/Activin, Bmp, PKA, FGF and Wnt signaling, were explored to determine if they could directly regulate LB specification.

### Retinoic Acid

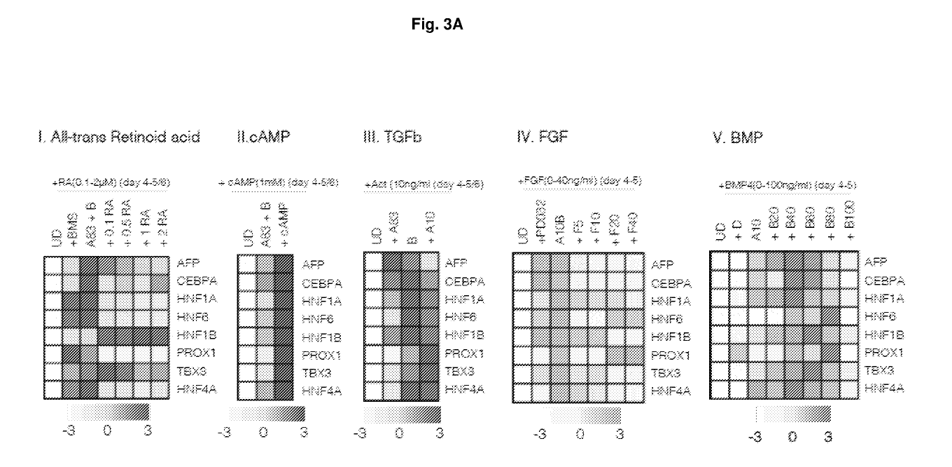

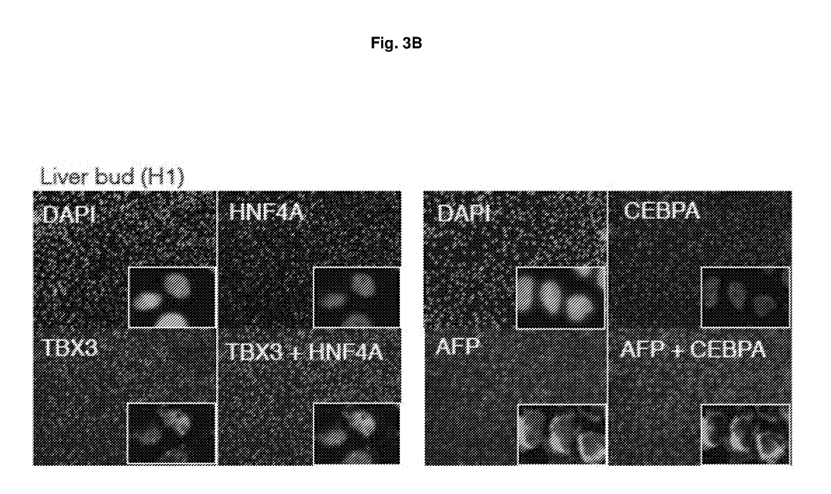

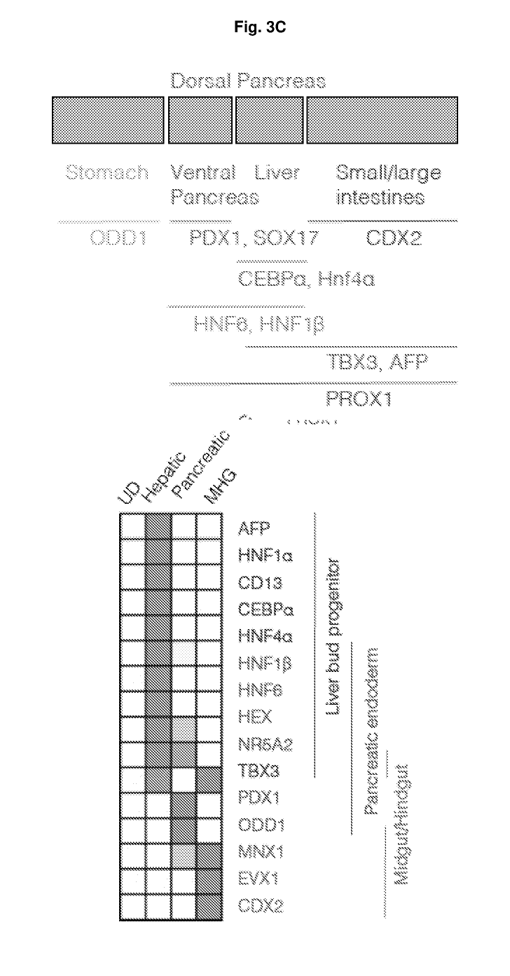

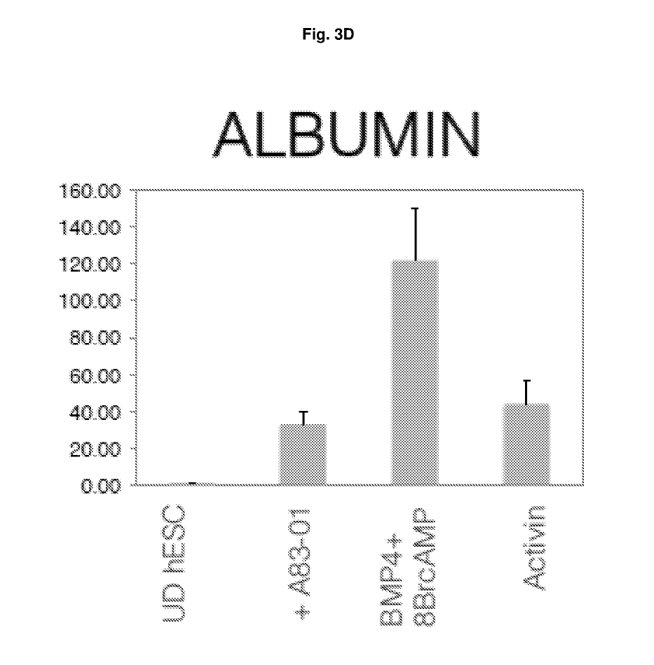

It has been demonstrated herein that early RA treatment promotes foregut competence to later generate liver cells, however, later RA treatment conversely inhibits LB specification and expression of AFP, CEBPA, HNF6 and HNF4A (FIG. 3). Thus, addition of RA at a later timing is detrimental to liver specification.

### BMP Signaling

It was found that BMP4 promotes LB but inhibited pancreatic specification (FIG. 2), also consistent with the outcome that liver specification was disabled in BMPR−/− mice. Furthermore, BMP4 maximally induces the expression of LB genes (HNF4A, PROX1, AFP, TBX3, HNF1B, HNF1A, HNF6, and CEBPA) at higher doses between 30-40 ng/ml. (FIG. 3).

### FGF Signaling

Inhibition of FGF/MAPK signaling by PD0325901 yielded reduced LB gene expression (HNF1A, HNF6, PROX1, TBX3, HNF4A), but the addition of FGF2 did not benefit LB gene expression of HNF1A, HNF6, PROX1, TBX3, HNF4A, AFP, CEBPA, HNF1B. This observation may be explained by the presence of existing FGF ligands either endogenous produced by cells or exogenously present in the CDM base media component knockout serum replacer (KOSR) (FIG. 3).

### TGFβ Signaling

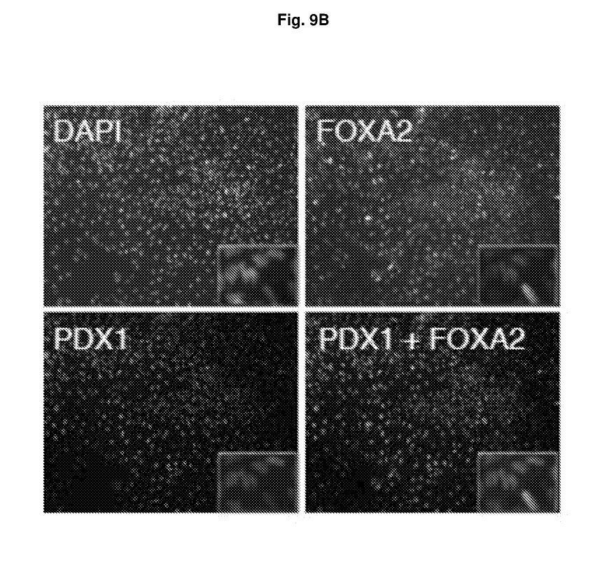

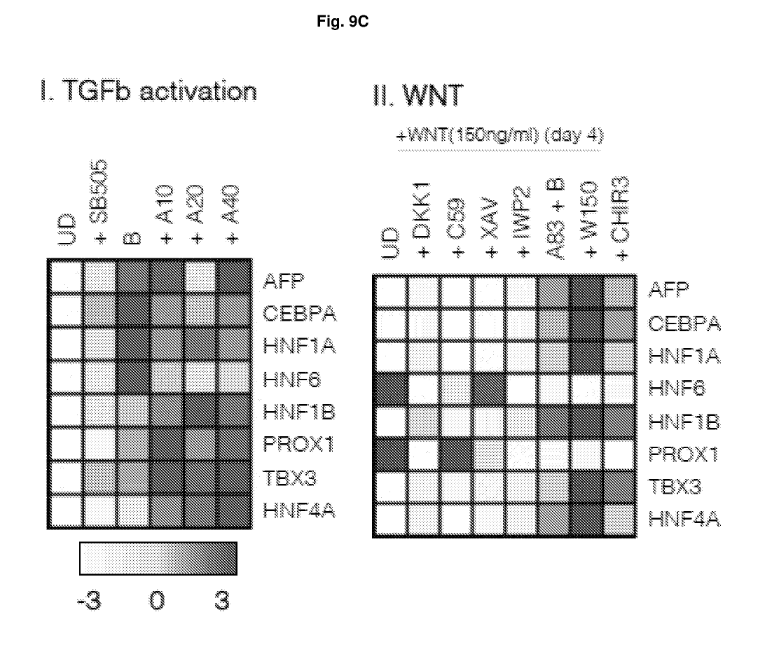

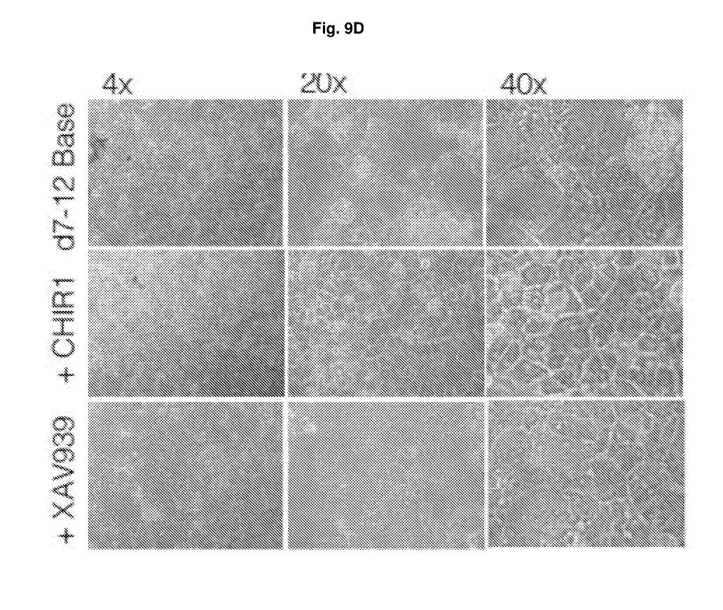

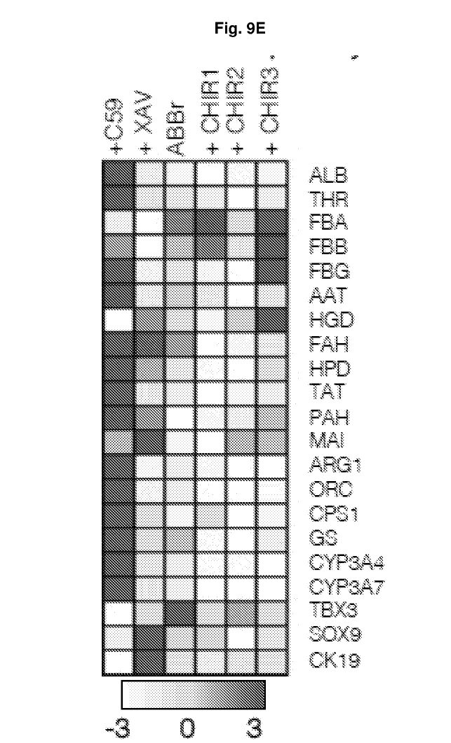

In mouse embryo, phospho-Smad2 staining was detected in LB cells at E10.5 suggesting active signaling has been induced then. Furthermore, through real-time PCR analyses it has been shown that TGFβ inhibition on day 4 to 6 affects different LB genes differently. For example, addition of Activin reduces LB expression (i.e. AFP and CEBPA), but increases HNF4A, HNF1A, and TBX3 levels (FIG. 3). The effect of Activin also appears to be dose dependent since increasing the dose of Activin seems to reduce LB marker expression (FIG. 9B). To decide whether Activin treatment in LB specification benefits the competence of the cells to later generate ALB-expressing liver cells, the cells were arbitrarily treated with hepatic specification medium.

Interestingly, TGFβ inhibition by A83-01 and SB505124 reduces the subsequent expression of ALB while the addition of Activin at 10 ng/ml increases the later expression of ALB expression in subsequent differentiation (FIG. 3D).

These results suggested that earlier treatment of Activin during LB specification promotes the competency of these cells to later express higher levels of ALB.

### Cyclic AMP/PKA Signaling

Prostaglandin E2 was shown to regulate endodermal specification into liver cells in the zebrafish during development and is also an upstream activator of PKA signaling. Apart from this, there is no other known role of PKA signaling in liver bud specification.

The treatment of cells with PKA activators, such as 8-bromoCAMP and Forskolin were shown to promote liver specification. Indeed, both treatments significantly increase expression of LB markers including AFP, CEBPA, HNF6, HNF1B, PROX1, TBX3 and HNF4A during LB specification (FIG. 3).

### Wnt Signaling

The dynamic and changing role of Wnt signaling during endoderm, foregut and liver specification in vivo necessitates temporally controlled activation of this pathway during in vitro differentiation.

In this regard, it was found that the early inhibition of Wnt signaling between days 3-6 during early liver bud specification resulted in stronger liver gene expression in hepatocyte-like cells. Shortly later the addition of Wnt3A or CHIR99201 at the LB specification stage promotes expansion and induces higher LB gene expression. (FIG. 9C, FIG. 9D).

These results indicated that PKA, BMP, Wnt, FGF TGFβ and RA play a role in early liver versus pancreatic specification. Precise demarcation of lineage boundary is reflected in the bounded expression of fate markers. For example, hepatic, pancreatic and MHG progenitors exclusively express liver bud progenitor, pancreatic endoderm and MHG markers respectively without cross contaminating Lineage expression. Specifically, gene expression of hPSC-derived LB, MHG and early pancreatic endoderm shows demarcation of gene expression boundary of key lineage specifiers or transcription factors such as HNF1A, CEBPS, HNF4A, HNF1B, HNF6, HEX, NR5A1, TBX3, PDX1, ODD1, MNX1, EVX1 and CDX2.

### Efficient Differentiation to Liver Bud (LB) Progenitors

DE was differentiated into PFG by treating it with RA, FGF, BMP4 while concomitantly inhibiting TGFβ signaling for 1 day (“BARF”). Subsequently, LB progenitors were specified from the PFG by treating it with Activin, and BMP for 3 days (“ActB”). This 2-step method can be referred to as “SR2”.

Together, this differentiation strategy resulted in highly homogeneous differentiation of AFP, CEBPA, HNF6, HNF1B, PROX1, TBX3 and HNF4A liver bud progenitors from diverse hESC lines including H1 (FIG. 3), H7, HES2 and HES3 (not shown), as confirmed by immunostaining.

The efficacy of method “SR2” was compared with existing hepatic-inducing conditions using RTqPCR. Starting from highly-pure DE, the DE was subsequently treated with differentiation factors of (i) SR2, (ii) BMP4 and FGF2 for the next 4 days or (iii) “FSB” which first induces foregut specification using FGF7 and SB for 2 days and next FGF2, FGF7, BMP4 for another 2 days.

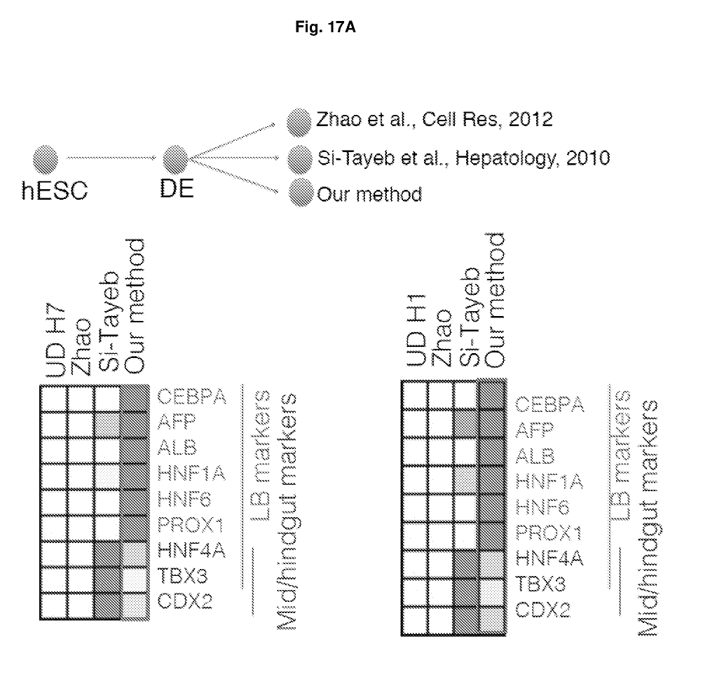

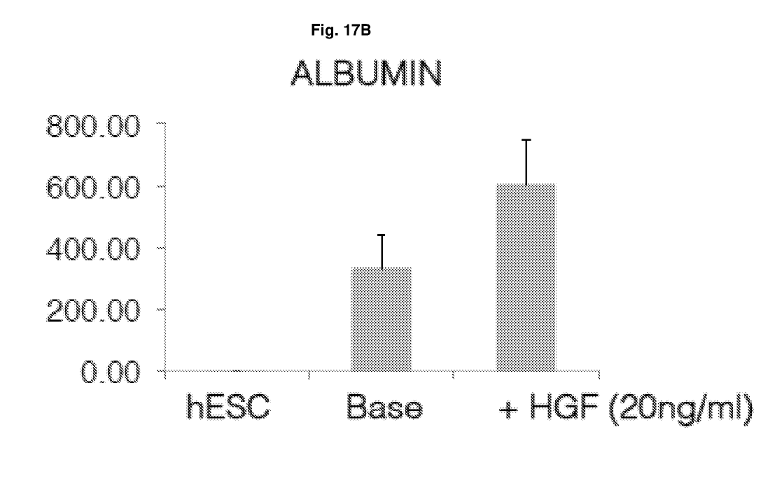

In this regard, qPCR revealed that method SR2 induced the highest expression of CEBPA, AFP, ALB, HNF1A, HNF6 and PROX1 by comparison to the other two approaches (disclosed in “Zhao, D et al. (2012). Promotion of the efficient metabolic maturation of human pluripotent stem cell-derived hepatocytes by correcting specification defects. Cell Res 23, 157-161”; and “Si Tayeb, K et al. (2010). Highly efficient generation of human hepatocyte-like cells from induced pluripotent stem cells. Hepatology 51, 297-305”). Conversely, MHG marker(s) CDX2 was upregulated in the populations induced from method (ii) described (FIG. 17).

In summary, the SR2 differentiation approach generates purer populations of LB progenitors than previous protocols; it further confirms the previously-emphasized importance of the signaling pathways involved in LB induction over this timeframe.

Identification of Cell-Type Specific Cell Surface Markers Specifically Expressed in hPSCs, Endoderm, LB Progenitors

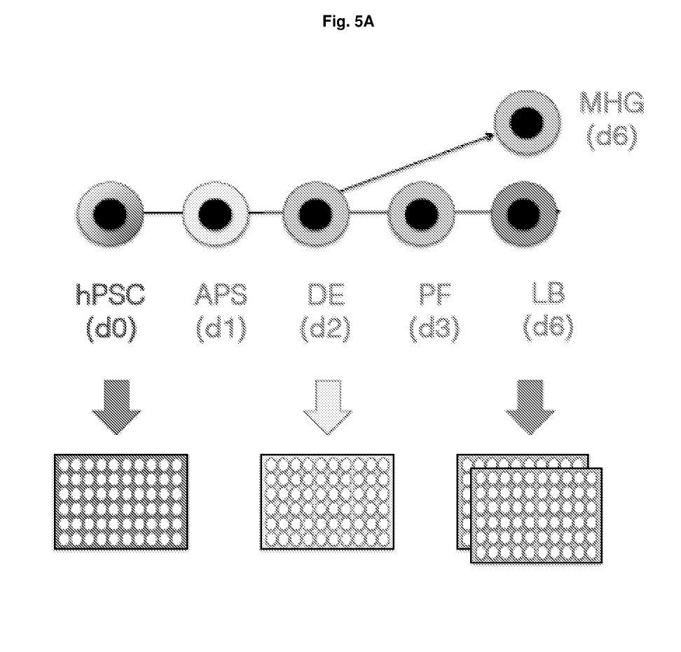

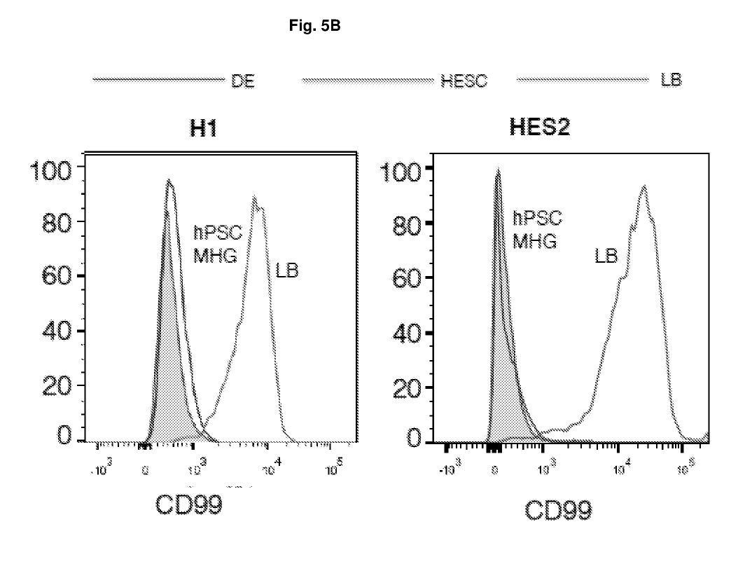

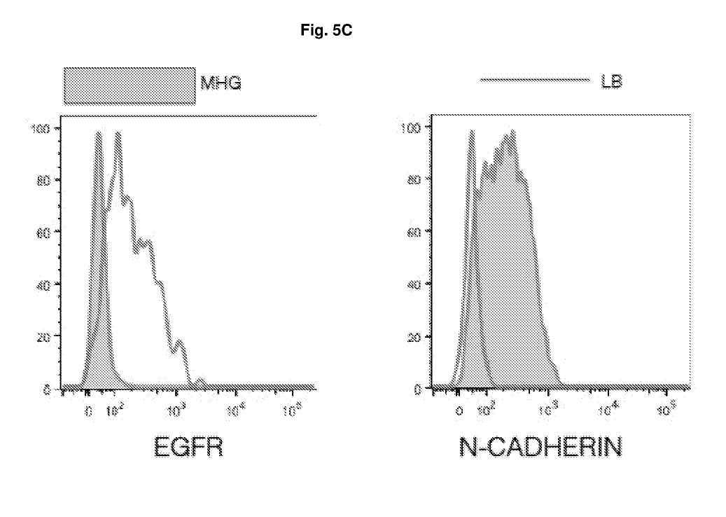

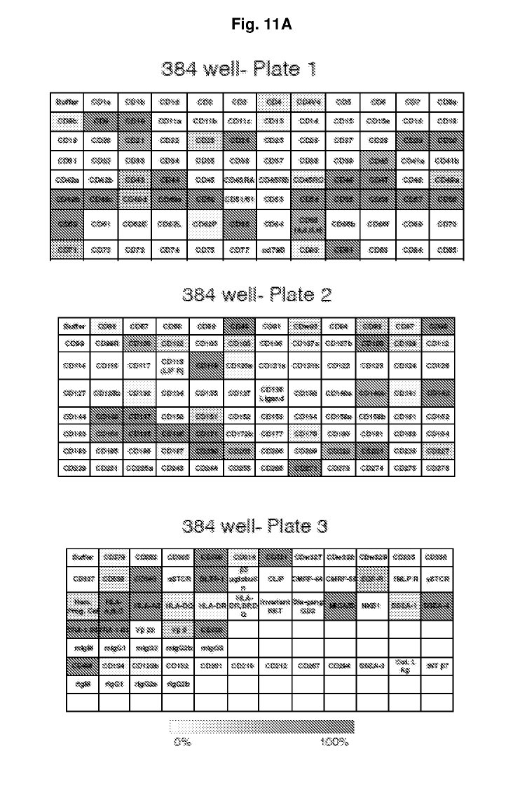

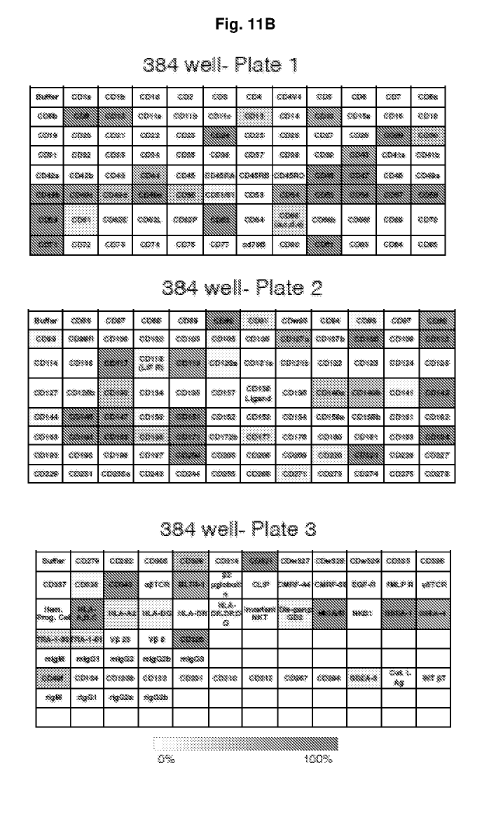

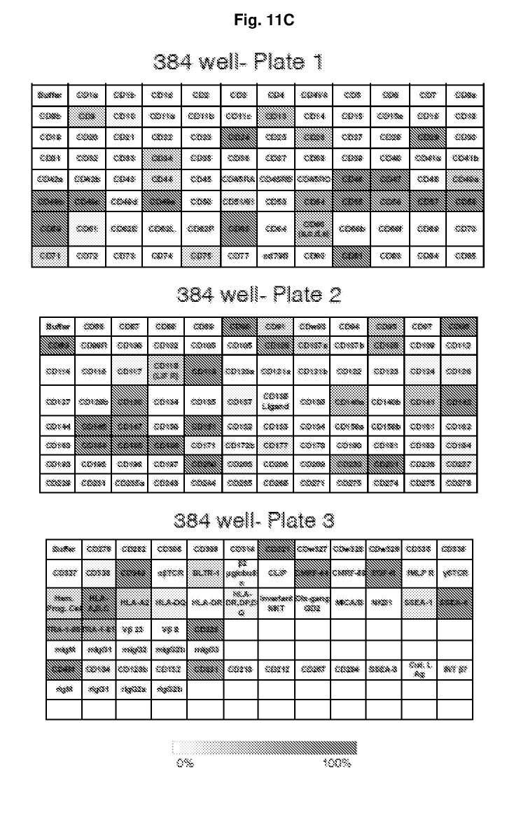

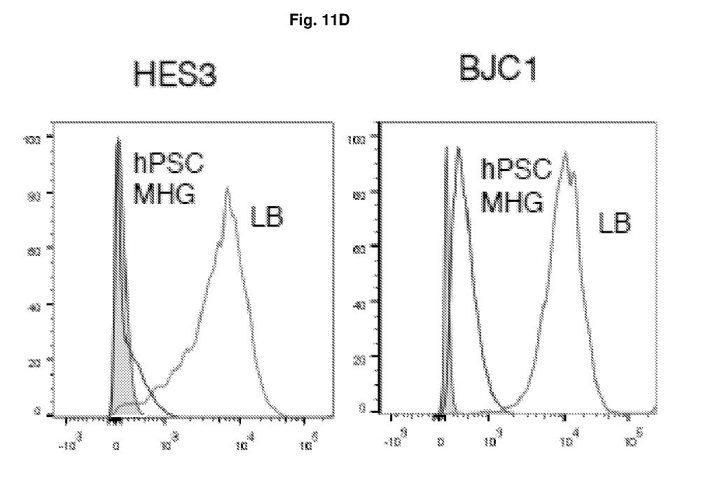

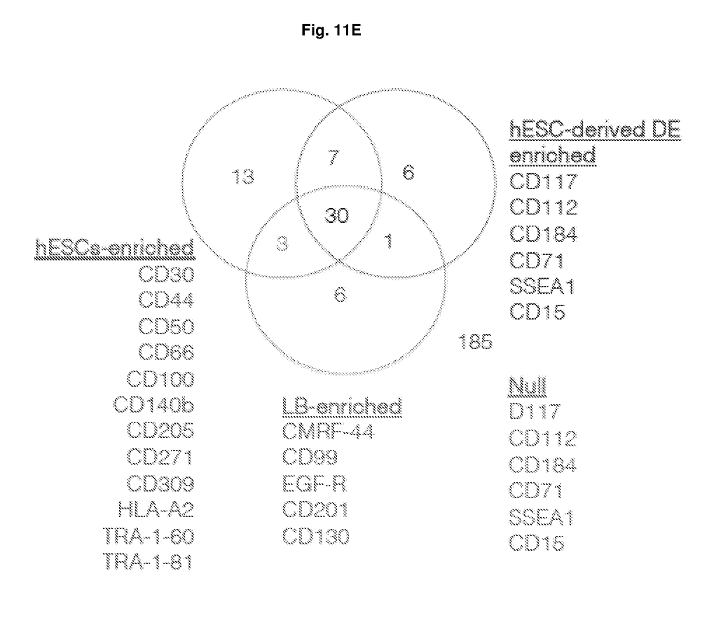

Human progenitors are difficult to access in vivo in large quantities and with high purity. Taking advantage of the abundant and rather-homogeneous hPSC, DE, LB and MHG populations generated in vitro, a high-throughput cell-surface marker screens were performed (242 surface markers and isotype controls). These analyses identified cell surface markers specific for each of the three developmental stages, for instance undifferentiated hPSC-enriched (CD21, CD30, CD44, CD50, CD66, CD100, CD140b, CD205, CD271, CD309, TRA-1-60 and TRA-1-81), DE-enriched (CD117, CD112 and CD184) and LB-enriched (CD99, CMRF44 and EGFR) markers (FIG. 5, FIG. 11). Though CD99 was present to some degree in other endodermal downstream derivatives (not shown), it was found that CD99 was ubiquitously expressed (>90%) in LB populations but was seemingly absent in the preceding hPSC and DE stages (FIG. 5, FIG. 11). This trend was consistent throughout the H1, H7, HES2, and HES3 cell lines, and suggests CD99 might help track cells at progressive stages of LB specification from hPSCs. (FIG. 5, FIG. 11).

In addition, to exclusively isolate and enrich for LB cells in absence of MHG cells, identification of cell surface markers were sought that were uniquely expressed on LB cells but not MHG cells. It was found that that CD325 is expressed on ˜75% MHG cells but not LB cells and EGFR is expressed on ˜70% of LB cells but absent on MHG cells (FIG. 5, FIG. 11).

III. Liver Bud (LB) Differentiation into Hepatic Progenitor

### FGF/MAPK, Notch, TGFβ, BMP, HGF and Wnt Signaling Mediates Hepatic Progenitor Versus Biliary Specification

ALB is not detected in liver buds at E9.5 but is expressed thereafter in E10.5 liver, suggesting a transition has occurred from the ALB-liver bud cells. Thus, the signaling conditions that promote ALB expression from the hPSC-derived LB cells were investigated.

To identify the signals that regulate the progression of the LB progenitors from an ALB- to a more differentiated ALB+ state, multiple signaling pathways were systematically screened for their effects on ALB expression (including FGF, WNT, Notch, HGF, cAMP/PKA, TGFβ, and BMP).

### Cyclic AMP/PKA Signaling

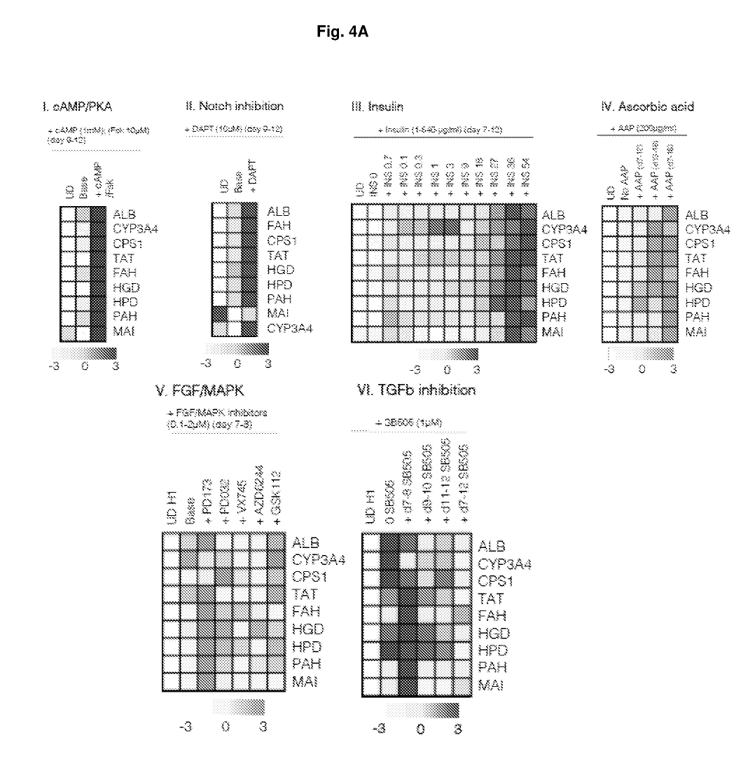

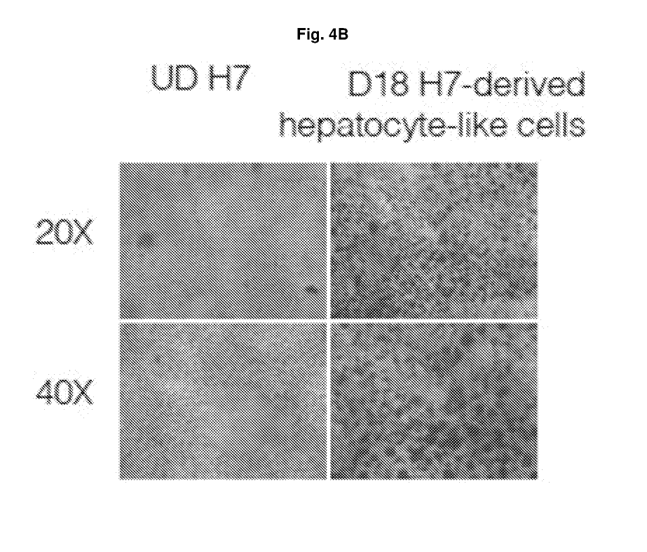

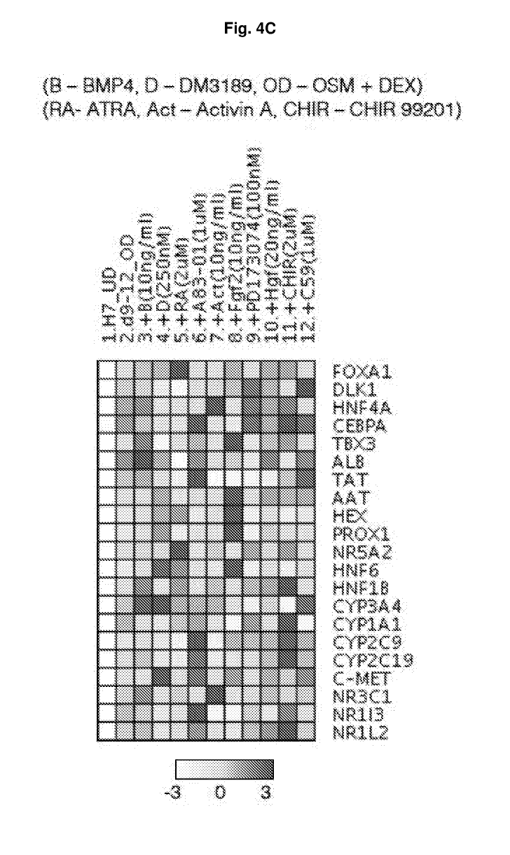

Treatment of liver bud progenitors with 8-BromoCAMP and forskolin on days 9-12 enhances expression of functional liver enzymes such as FAH, PAH, HGD, HPD, TAT, CPS1, CYP3A4 and promotes hepatic progenitor markers ALB expression (FIG. 4A).

### BMP Signaling

The addition of BMP4 to the LB cells was found to promote ALB expression in hepatic progenitor, and inhibition of BMP signaling conversely strongly abrogates expression of liver genes (FIG. 4A).

### TGFβ Signaling

It was found that the prolonged treatment of liver cells (beyond LB stage) between days 7-12, days 9-12 or days 7-10 with SB505124 reduces competence of hepatic progenitors to generate hepatocyte-like cells and results in lower expression of liver genes such as ALB, CYP3A4, CPS1, FAH, TAT, PAH, HGD, HPD and MAI. On the other hand, brief SB505124 treatment on days 7-8 led to later higher functional liver gene expression of TAT, FAH, PAH, HGD, HPD, and MAI (FIG. 4A). In other words, brief TGFb inhibition generally induced hepatic progenitors competent to differentiate to downstream hepatocyte-like cells.

### Wnt Signaling

Wnt signaling plays a temporally dynamic role during liver specification, first it was repressed in foregut, and subsequently it was activated during LB specification. Thereafter, ectopic activation of canonical wnt signaling in the liver bud by APC inactivation/beta-catenin activation led to hypoplastic mouse liver, blockade of ALB expression, and failed hepatocyte differentiation.

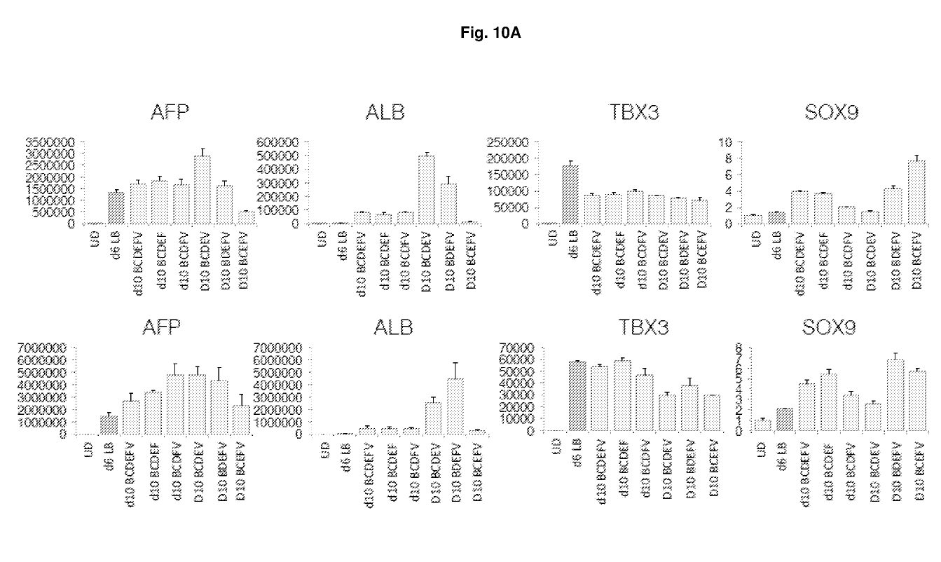

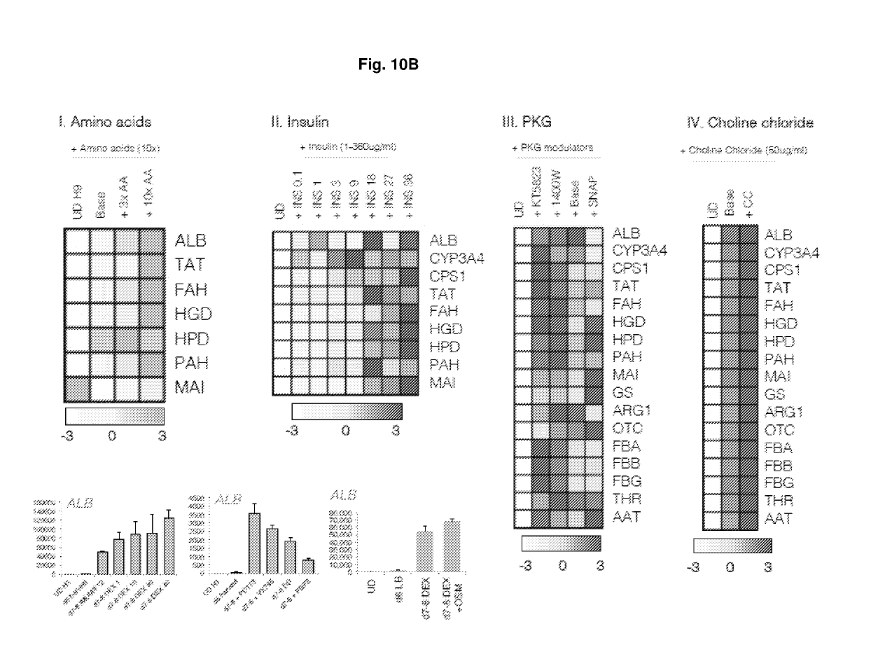

In this regard, it is shown that early Wnt inhibition during early foregut or LB specification (days 4-6) promotes downstream hepatocyte specification (FIG. 9E) while Wnt activation promotes liver bud expansion and specification (FIG. 9D), however, later Wnt activation of the LB progenitors inhibits their ALB expression and progression to the hepatic fate (FIG. 10).

Thus, the removal of Wnt agonists between day 7-10 strongly enhances ALB expression, indicating that the Wnt pathway inhibits ALB expression (FIG. 10).

### FGF Signaling

It is shown that FGF2 treatment (20 ng/ml) of the liver bud progenitors resulted in reduction of ALB expression (FIG. 10) and also appeared to slightly increase SOX9 expression (FIG. 10), suggesting a role in segregating hepatic versus biliary fate choice.

This result is consistent with the finding that FGF promotes biliary specification of chick hepatoblast explants. Conversely, brief treatment of LB cells with FGF inhibitor PD173074 or MAPK inhibitor GSK1120212 for 2 days resulted in higher subsequent expression of liver genes (FIG. 4AV). These results suggested that the inhibition of FGF signaling prompted the exit of the LB progenitor state.

As such, the removal of FGF agonists between day 7-10 strongly enhances ALB expression, indicating that the FGF pathway inhibits ALB expression.

### Notch Signaling

Jagged-1 is a Notch ligand expressed in the portal endothelium near liver cells at E12.5 while Hes 1, a Notch target gene, is expressed at E14.5. Conversely, activation of Notch during liver development results in ectopic biliary specification. Even in postnatal hepatocytes, Notch activation results in their conversion to a biliary fate.

Notch inhibition by DAPT or R04929097 was shown to enhance ALB while reducing SOX9 expression (FIG. 4A).

Thus, Notch inhibitor treatment on the LB also enhances ALB expression and seems to reduce SOX9 expression, mirroring previous results from reprogrammed liver cells and liver organoids. This suggests that Notch inhibition drives the progression of the LB progenitors to the next hepatic progenitor state.

Therefore, a simultaneous blockade of Notch (DAPT or R04929097) pathways was employed to drive day 6-TBX3+LB progenitors to a later ALB+ hepatic precursor state.

### HGF Signaling

In mouse embryos, transcripts of cMet and HGF are detected by E11. In the adult mouse, HGF signaling plays a role in the regeneration of hepatocytes upon partial resection of liver. The addition of HGF to primary cultures of E14 mouse liver cells also promoted their maturation. Accordingly, it was discovered that the addition of HGF to the LB-like cells promotes the expression of ALB (FIG. 17B).

### OSM and DEX Addition Promote ALB Expression and TBX3 Downregulation

While progression from LB stage to the ALB+ stage is enhanced by the addition of Notch inhibitors and by the treatment with HGF, it was found that OSM and DEX also promoted ALB expression (FIG. 10).

IV. Developmental Signals that Promote Liver Maturation Also Promote Maintenance of Adult Human Liver Phenotype

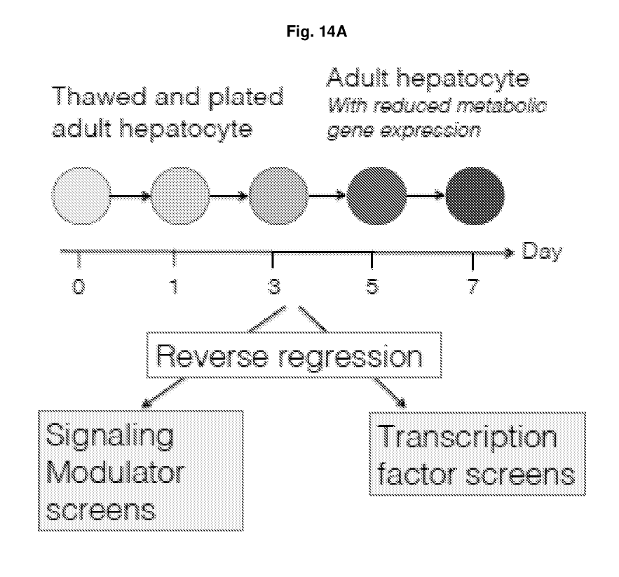

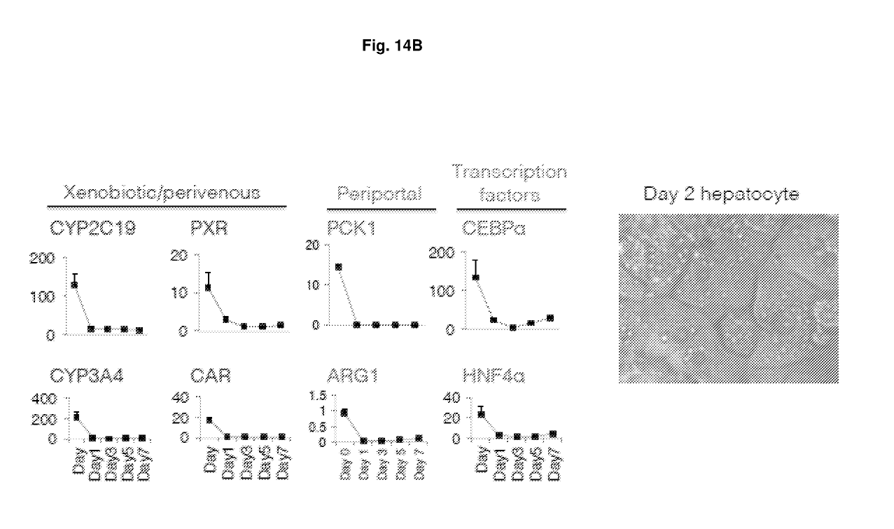

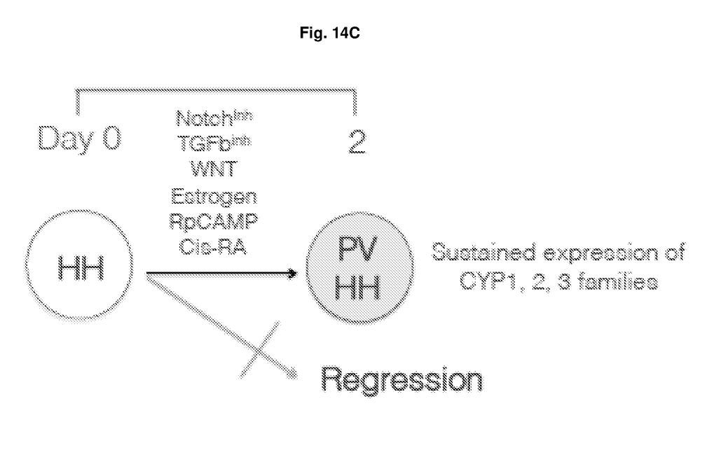

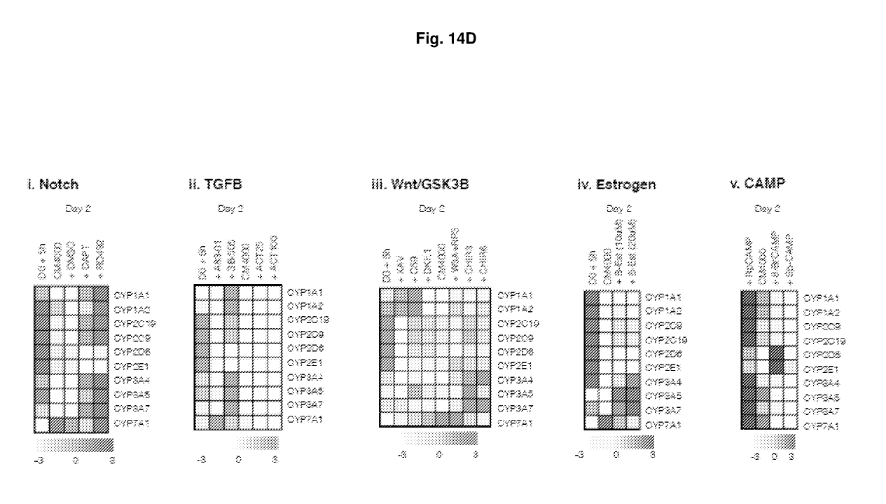

Primary human hepatocytes are known to regress during in vitro culture and lose expression of their xenobiotic enzymes. Accordingly, gene expression profiling was carried out on human hepatocytes during in vitro culture using commercial medium over a course of 0, 1, 3, 5, and 7 days by RNA-seq. A sharp decline of many functional important hepatic genes was observed. QPCR validation confirmed that protein metabolic enzymes (ARG1), carbohydrate metabolic enzymes (PCK1) and xenobiotic regulators and P450 cytochrome enzymes (PXR, CAR, CYP2C19, CYP3A4) were downregulated sharply after 1 day in culture (FIG. 14).

Since spontaneous loss of liver function has not been observed in vivo, it was hypothesized that the extrication from the signaling environment in the liver could have resulted in the dysregulation of hepatic gene expression and loss of liver maturity. However, no significant increase in expression of SOX9 and AFP was detected during the loss of phenotypic maturity of the hepatocytes. Moreover, gene expression of components of signaling pathways such as RBPJ (Notch mediator), SMAD2 (TGFb mediator), WNT4 (Wnt ligand), WNT11 (Wnt ligand) appears to be dysregulated. For example, RBPJ and SMAD2 expression increases, while WNT4 and WNT11 expression becomes down regulated.

The signals that promote hepatocyte specification and/or maturation were considered to also ameliorate such loss of liver maturity and gene expression during in vitro culture. Thus, the signaling pathways that appear to be dysregulated were investigated including Notch, TGFb, Wnt, as well as other major signaling pathways including estrogen, BMP, FGF, PKA/cAMP pathways, in order to determine the effects on liver genes after 2 days.

The expression of cytochrome enzymes (e.g. CYP1A1, CYP1A2, CYP2C19, CYP2C9, CYP2D6, CYP2E1, CYP3A4, CYP3A5, CYP3A7, and CYP7A1) was found to be downregulated 2 days after culturing in standard media. However, Notch inhibition by small molecule signaling inhibitors (DAPT and RO4929097) upregulated the expression of the majority of the aforementioned cytochrome enzymes (FIG. 14) to levels comparable to after the hepatocytes were just thawed for 5 hours. Activation of Notch in hepatocytes converts them into the biliary fate.

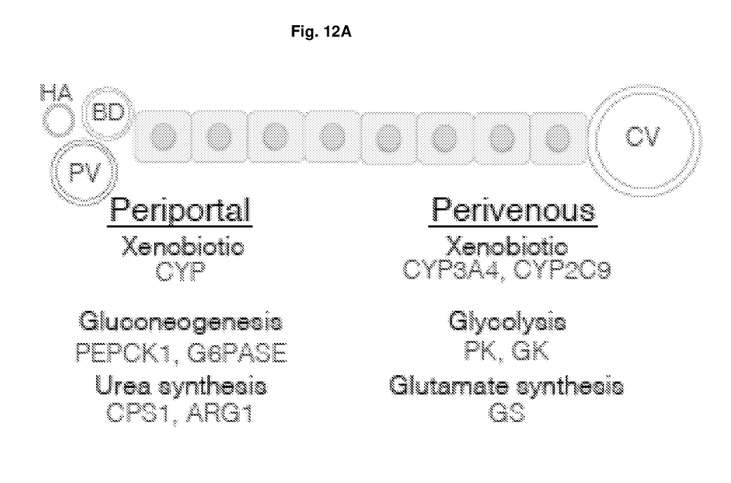

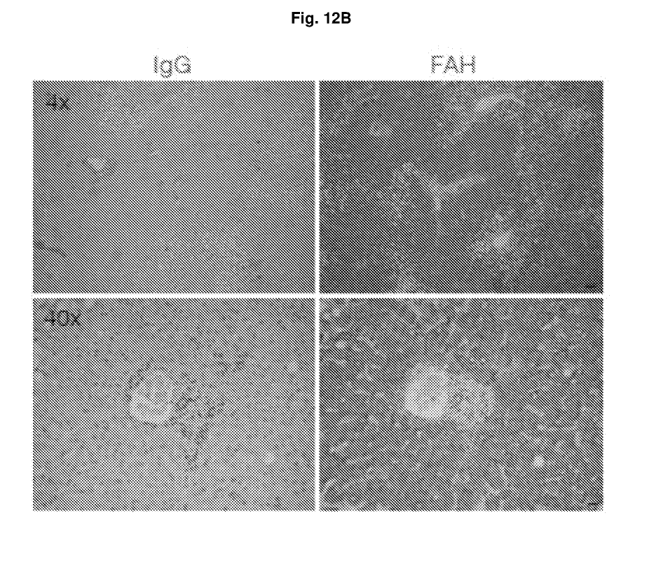

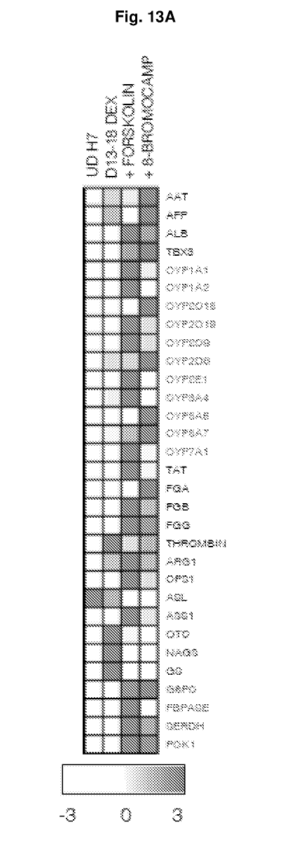

Similarly, TGFβ inhibition by A83-01 or SB-505124 increased the expression of CYP1A1, CYP1A2, CYP2C19, CYP2C9, CYP3A4, CYP3A5, CYP3A7 and CYP7A1 (FIG. 11). Notably, the effect of SB-505124 on the cytochrome expression appears stronger than A83-01. Specifically, it was shown that TGFβ promotes perivenous over periportal specification, whereby Activin was shown to promote CYP3A4 and CYP3A7 expression in a dose dependent fashion, with greater increase at higher dosage (e.g. 100 ng/ml). This is surprising given that TGFβ promotes biliary fate specification during liver development. On the other hand, Activin treatment appears to result in downregulation of periportal genes (FIG. 12). TGFβ inhibition also enhances ABCC2 and ABCC3 expression (FIG. 14).

In adult mice, Wnt signaling has been demonstrated to regulate the zonated expression of protein metabolic enzymes. In the present invention, it was discovered that Wnt3a/GSK3b treatment increased perivenous xenobiotic gene expression (e.g. CYP3A4, CYP3A5 and CYP3A7) (FIG. 14). GSK3 inhibition/Wnt activation promotes expression of apical and basal transporters including ABCC2, ABCC3, and ABCB11.

Moreover, Wnt inhibition by small molecule C59 or Dkk1 on d13-18 promotes the expression of ALB, CYP3A7 (FIG. 14). Conversely, the addition of GSK3 inhibitor (CHIR99201) vastly reduces the expression of hepatic genes (FIG. 14).

During differentiation of hepatocytes from hPSCs, PKA/cAMP signaling plays a major role in increasing liver gene expression of ALB, CYP3A4, and tyrosine metabolic genes.

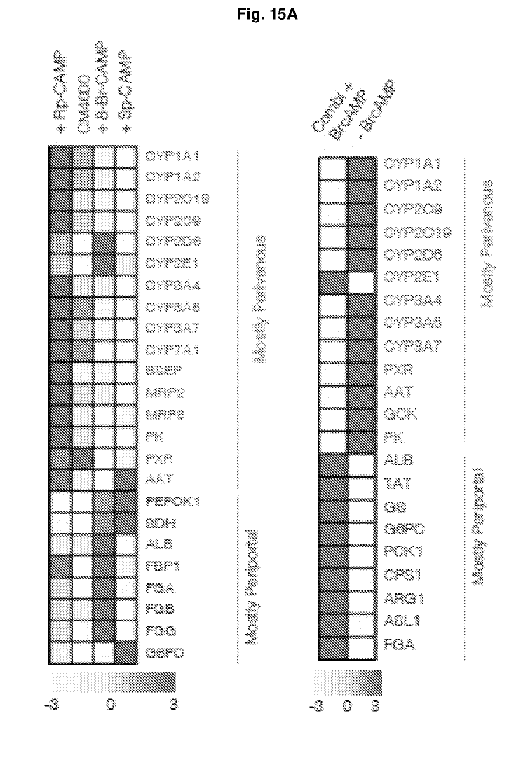

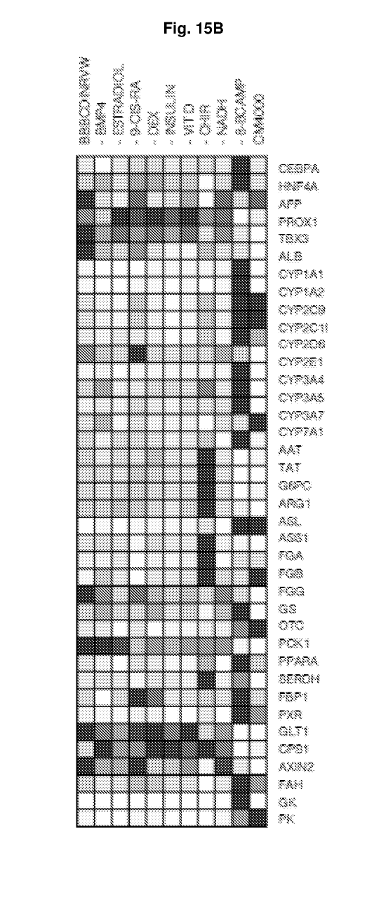

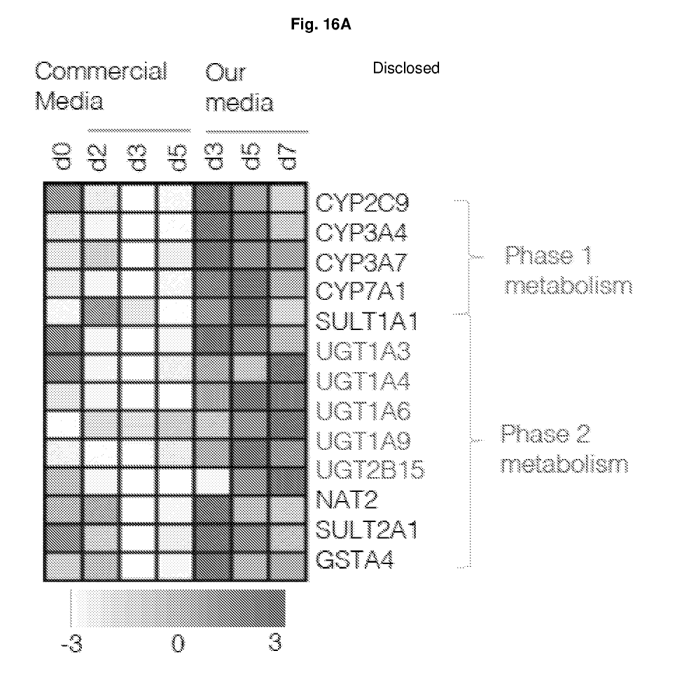

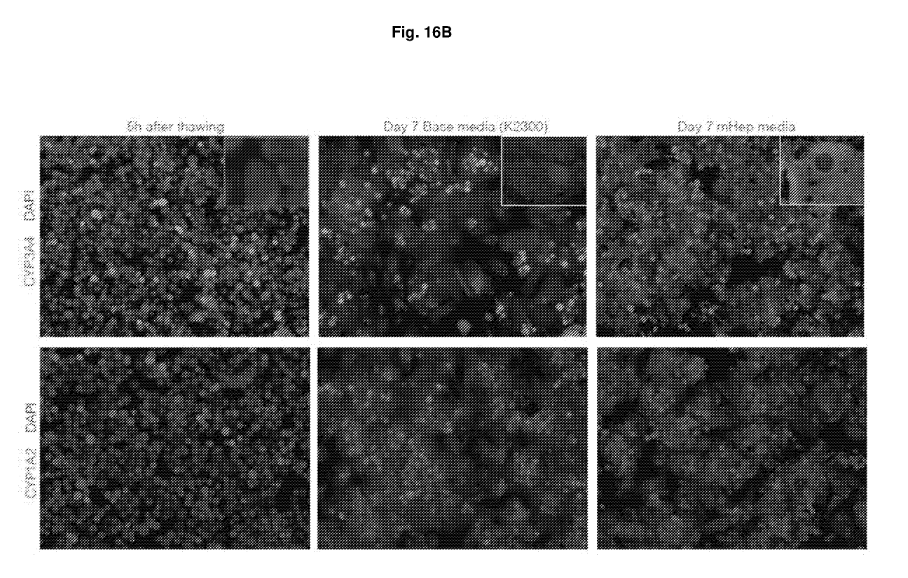

In adult primary hepatocytes, PKA/cAMP agonists, such as 8-BromoCAMP and spCAMP promote periportal gene expression whilst reducing perivenous gene expression (e.g. members of the CYP1, CYP2 and CYP3 families). Conversely, cAMP antagonist RpCAMP promoted p450 cytochrome expression (FIG. 15). In this regard, it was shown that PKA agonism increases expression of periportal genes such as CPS1, ARG1 and G6P whilst concomitantly decreasing perivenous gene expression. This supports that PKA/cAMP activation promotes the lineage segregation of periportal lineage over perivenous fate.

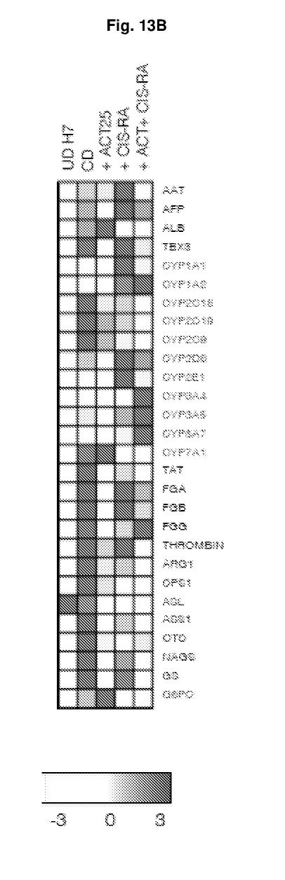

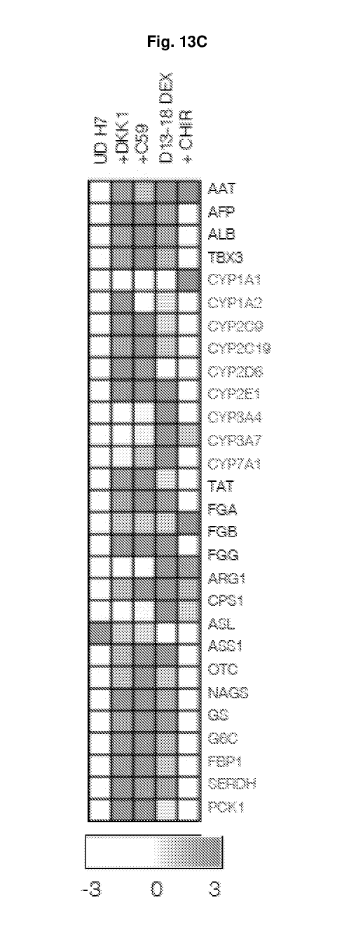

In addition, vitamin derivative, 9-Cis-RA was shown to enhance the expression of CYP3A5, CYP3A7, CYP3A4, CYP2D1, CYP2E1, and CYP1A1 whilst conversely reduces the expression of periportal genes such as ARG1, OTC, ASL1 and CPS1 (FIG. 13).

PKG signaling was also shown to regulate the periportal and perivenous gene expression. Treatment of hepatic progenitors with PKG antagonists, 1400 W dihydrochloride and KT5823 both upregulate expression of FAH, PAH, HGD, TAT, CPS1, ARG1, FBA, FBB and FBG. On the other hand, PKG agonist S-nitrosoacetyl penicillamine (SNAP) treatment results in decrease of FAH, CYP3A4 and increase of GS expression (FIG. 10).

Insulin and Ascorbic Acid Treatment of Hepatic Progenitors or Liver Cells Augments Expression of Tyrosine Metabolic Genes in hPSC-Derived Hepatocyte-Like Cells

One key function of the liver is to break down toxic byproducts of protein metabolism known as tyrosine metabolites. Liver metabolic disorders including hereditary tyrosinemia type 1 and tyrosinemia type 2 results from deficiency of enzymes such as Fumarylacetoacetate hydrolase (FAH) and tyrosine aminotransferase (TAT). The tyrosine metabolic pathway involves a series of enzymes including PAH, HGD, HPD, TAT, MAI, and FAH.

As such, the signaling factors that could induce significantly high expression of these tyrosine metabolic genes in the hPSC-derived liver cells were investigated.

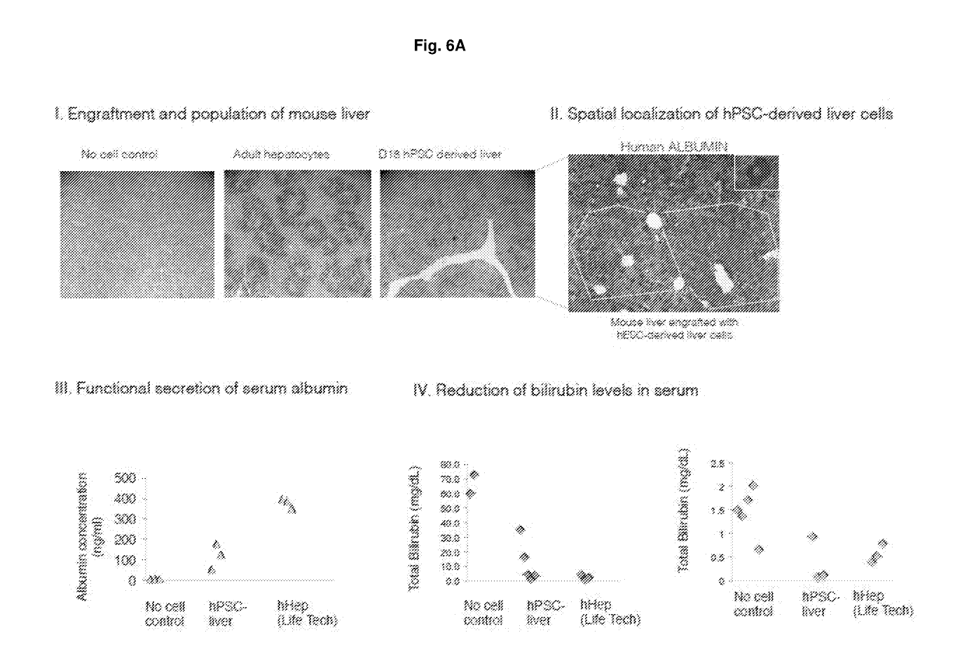

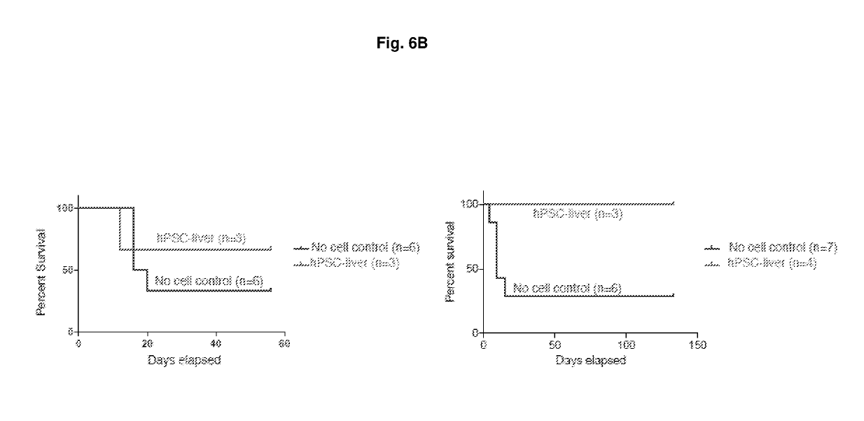

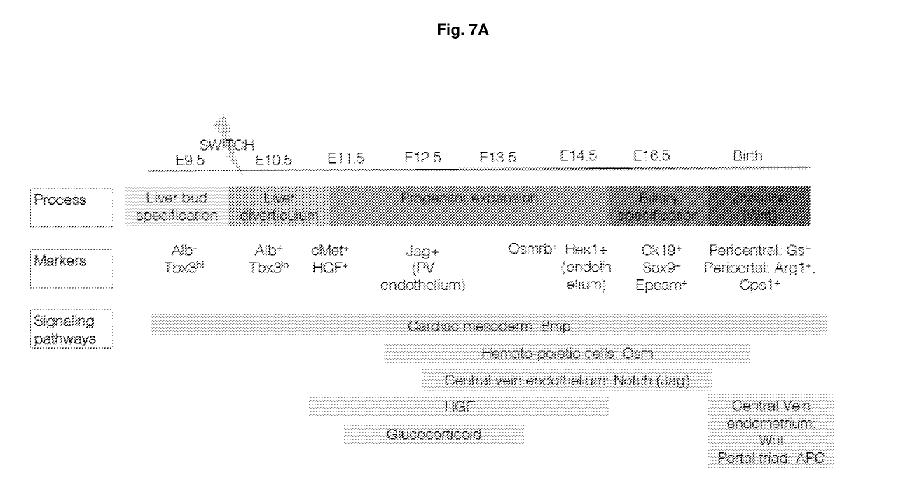

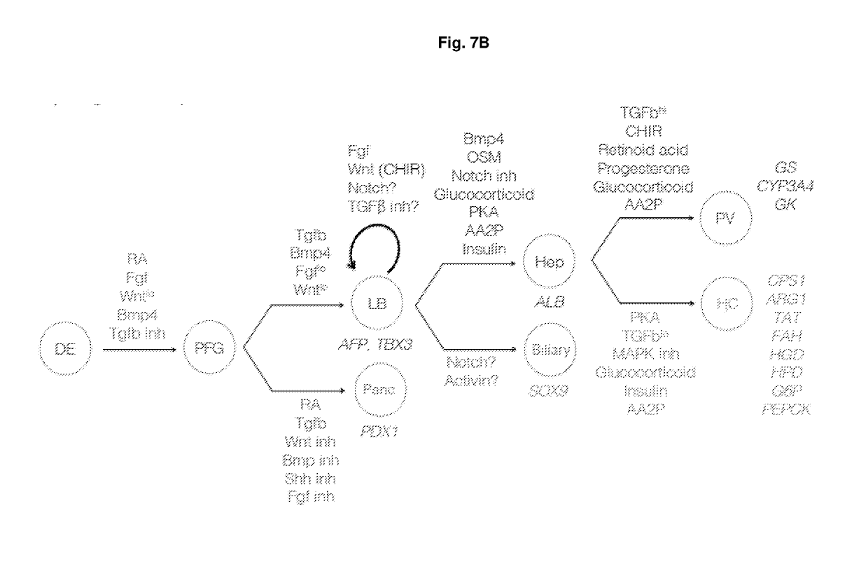

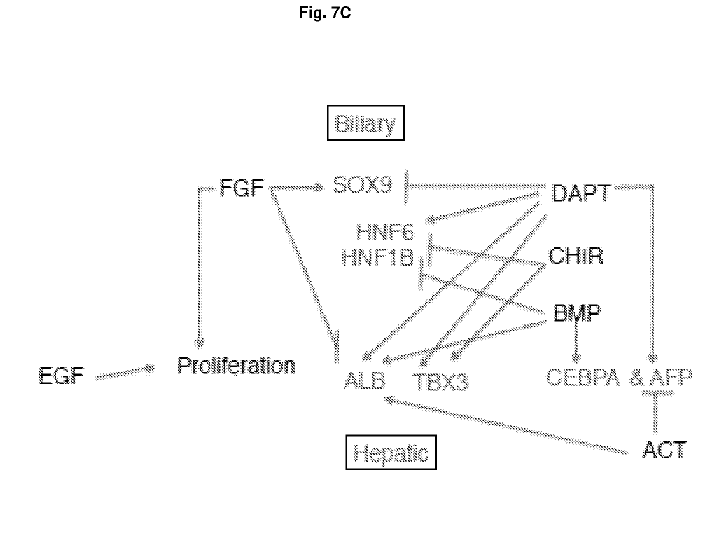

Strikingly, it was found that human recombinant insulin treatment between days 7 to 12 during hepatic specification significantly increases the expression of functionally important liver genes including PAH, HGD, HPD, TAT, MAI, and FAH (FIG. 4). Other genes such as ALB, CPS1 and CYP3A4 are also strongly enhanced (FIG. 6). Subsequent treatment of insulin from days 13 to 16 continues to enhance tyrosine metabolic genes and xenobiotic metabolic genes such as CYP3A4 (FIG. 4).

The treatment of liver bud progenitors with L-Ascorbic acid-2-Phosphate (AA2P), a more stable form of ascorbic acid, between days 7 to 12 and at a later stage during days 13 to 16 also significantly promoted liver maturation. For example, enhancement of the expression of PAH, HGD, HPD, TAT, MAI, FAH, CYP3A4, ALB and CPS1 is observed with AA2P treatment (FIG. 4).

Moreover, the addition of amino acids between days 7 to 12, but not days 13-16, was shown to promote liver gene expression (FIG. 10), suggesting that heightened amino acid levels benefits liver maturation.

Liver maturation and attainment of complex metabolic functions require higher levels of phospholipids, which are building blocks for membrane-rich organelles such as endoplasmic reticulum, golgi apparatus, mitochondria and lysosome. Hence, it was investigated if the supply of phospholipid precursors would promote liver maturation. Two major types of phospholipids are phosphatidylcholine (˜50% of total phospholipids) and phosphatidylethanolamine (˜20-30% of phospholipids). It was found that provision of higher levels of phospholipid precursor choline chloride increases the expression of ALB, FAH, PAH, TAT, CYP3A4, CYP3A7, AAT, THR, FBA, FBB, FBG and TBX3. (FIG. 10).

Repopulation of Liver by hPSC-Derived Hepatic Progenitors

Generating of hPSC-derived hepatic progenitors that truly engraft, proliferate and repopulate host liver and restore liver function remains a challenging goal. The FRG−/− genetic mouse model has been widely used to induce injury in native FAH−/− liver cells and hence provide selection advantage for FAH+ liver cells. To test the authenticity of the FAH-expressing hESC-derived hepatic progenitors, the cells were transplanted intraspenically into the FRG−/− mice, and chronic liver injury was induced by NTBC withdrawal and then examination of the i) degree of repopulation, ii) localization of cells, iii) secretion of human serum albumin, iv) degree of bilirubin levels v) rate of survival and vi) expression of ALB and FAH was conducted.

H9 and H7 hESCs ectopically expressing antiapoptotic gene BCL2 and wildtype H1 hESCs are used as sources of hESCs to generate hepatic progenitors for transplantation. Adult human cryopreserved hepatocytes and media lacking cells are intrasplenically injected into mice for positive and negative controls respectively.

3 months after transplantation, human ALB-expressing liver cells were detected in the mouse liver, indicating integration of hPSC-derived liver cells in the mouse liver. These human cells appear to be dispersed throughout the entire liver lobules and localized near vasculatures including the portal and central veins (FIG. 6). The dispersion of these cells in the liver near blood vessels and sinusoids may facilitate secretion of proteins such as Albumin (FIG. 6).

In particular, ˜10-200 ng/ml human albumin was detected in the mice serum (n=7) indicating secretion by the transplanted liver (FIG. 6) and reduction of bilirubin levels in the serum of these mice (FIG. 6). The survival of the mice transplanted with hPSC-derived liver cells is improved compared to the no-cell injected controls (FIG. 6).

Together, these results indicate functional integration of the transplanted liver cells into the host organ and amelioration of liver injury.

Characterization of hPSC-Derived LB and Hepatic Progenitors

A lyoplate screen is conducted on hPSC-derived LB cells (day 6) and hPSC-derived mid/hind gut (MHG) cells. It was shown that CD325 is expressed on ˜75% MHG cells but not LB cells and EGFR is expressed on ˜70% of LB cells but absent on MHG cells (FIG. 5, FIG. 11).

1. A method of differentiating cells of the definitive endoderm (DE) lineage into posterior foregut lineage comprising contacting said stem cells with: one or more retinoic acid activators; and/or one or more inhibitors of TGFβ signaling, optionally further comprising contacting said stem cells with one or more activators of BMP signaling, optionally further comprising contacting said stem cells with one or more activators of FGF signaling, optionally wherein the cells of the posterior foregut lineage comprise elevated gene expression of posterior foregut lineage markers and decreased expression of dorsal foregut markers relative to undifferentiated cells, optionally wherein the duration of the method is about 1 to 84 hours.

2.-5. (canceled)

6. A method of differentiating cells of the posterior foregut lineage into liver bud progenitors comprising contacting said cells of the posterior foregut lineage with: one or more activators of TGFβ signaling, one or more modulators of Wnt signaling; and/or one or more activators of cyclic AMP/PKA signaling, optionally further comprising contacting said cells of the posterior foregut lineage with one or more activators of BMP signaling, optionally wherein the liver bud progenitors comprise elevated gene expression of liver bud progenitor markers and decreased expression of pancreatic progenitor markers relative to undifferentiated cells, optionally wherein the liver bud progenitors comprise elevated gene expression of markers comprising AFP, TBX3, HNF4A PROX1, HNF1B, HNF6, CEBPAα or a combination thereof, relative to undifferentiated cells, optionally wherein the one or more modulators of Wnt signaling comprise an inhibitor of Wnt signaling and/or an activator of Wnt signaling, optionally wherein the inhibitor of Wnt signaling is contacted with the cells of the posterior foregut lineage for duration of about 1 to 72 hours, and subsequently the activator of Wnt signaling is contacted with the cells of the posterior foregut lineage for duration of about 24 to 48 hours, optionally wherein the duration of the method is for about 1 to 120 hours, optionally wherein said cells of the posterior foregut lineage are obtained from the method of claim 1.

7.-13. (canceled)

14. A method of differentiating liver bud progenitors into hepatic progenitors comprising: contacting said liver bud progenitors with: one or more inhibitors of TGFβ signaling; one or more inhibitors of FGF signaling; and/or one or more inhibitors of Notch signaling, optionally further comprising contacting the cells with: one or more inhibitors of Notch signaling; one or more activators of ascorbic acid signaling, one or more activators of cyclic AMP/PKA signaling; and/or one or more activators of insulin signaling, optionally wherein the duration of contact is for about 48 hours, optionally wherein the duration of contact is for at least 1 to 108 hours, optionally further comprising contacting said liver bud progenitors with one or more differentiation factors selected from the group comprising of: activators of ascorbic acid signaling; activators of glucocorticoid signaling; activators of cyclic AMP/PKA signaling; activators of insulin signaling; activators of oncostatin M signaling; an amino acid mixture, and/or activators of L-glutathione signaling, optionally further comprising contacting the cells with one or more differentiation factors selected from the group comprising of: activators of BMP signaling; inhibitors of PKG signaling; activators of glucocorticoid signaling; phospholipid precursors; activators of oncostatin M signaling an amino acid mixture, and activators of L-glutathione signaling, optionally wherein the hepatic progenitors comprise elevated gene expression of hepatic markers and decreased expression of biliary markers relative to undifferentiated cells, optionally wherein the hepatic progenitors comprise elevated gene expression of markers comprises ALBUMIN, c-MET, HNF4A, CEBPA or a combination thereof, relative to undifferentiated cells, optionally wherein the hepatic progenitors comprise decreased gene expression of biliary marker SOX9 relative to undifferentiated cells, optionally wherein said liver bud progenitors are obtained from the method of claim 6.

15.-23. (canceled)

24. A method of differentiating hepatic progenitors into perivenous hepatocyte-like cells comprising contacting said hepatic progenitors with: one or more inhibitors of Notch signaling, one or more activators of glucocorticoid signaling, one or more activators of insulin signaling; one or more activators of ascorbic acid signaling, and/or one or more activators of TGFβ signaling, optionally further comprising contacting the hepatic progenitors with one or more activators of retinoic acid signaling, optionally further comprising contacting said hepatic progenitors with one or more activators of progesterone signaling, optionally further comprising contacting said hepatic progenitors with one or more activators of vitamin D signaling, optionally further comprising contacting said hepatic progenitors with one or more activators of PKG signaling, optionally wherein the perivenous hepatocyte-like cells comprise elevated gene expression of perivenous hepatocytes markers and decreased expression of hepatocyte-like cell markers relative to undifferentiated cells, optionally wherein the perivenous hepatocyte markers comprise GS, CYP3A4, AAT, AXIN2 or a combination thereof, optionally wherein the duration of the method is about 1 to 168 hours, optionally wherein said hepatic progenitors are obtained from the method of claim 14.

25.-32. (canceled)

33. A method of differentiating hepatic progenitors into hepatocyte-like cells comprising contacting said hepatic progenitors with: one or more activators of cyclic AMP/PKA signaling, one or more activators of glucocorticoid signaling; one or more activators of insulin signaling; one or more activators of ascorbic acid signaling, and/or one or more inhibitors of Notch signaling optionally further comprising contacting said hepatic progenitors with one or more modulators of Wnt signaling, optionally further comprising contacting said hepatic progenitors with one or more inhibitors of PKG signaling, optionally wherein the hepatocyte-like cells comprise elevated gene expression of hepatocyte-like cell markers and decreased expression of perivenous markers relative to undifferentiated cells, optionally wherein the hepatocyte-like cell markers comprise FAH, PAH, HGD, HPD, PAH, TAT, Albumin, AAT, ARG1, CPS1 or a combination thereof, optionally wherein the duration of the method is for about 1 to 168 hours, optionally wherein said hepatic progenitors are obtained from the method of claim 14.

34.-39. (canceled)

40. A method of maintaining liver bud progenitors in a self-renewal state comprising contacting said liver bud progenitors with: one or more activators of FGF signaling; and/or one or more activators of Wnt signaling, optionally further comprising contacting said liver bud progenitors with one or more activators of BMP signaling, optionally further comprising contacting said liver bud progenitors with one or more epidermal growth factors, optionally further comprising contacting said liver bud progenitors with one or more modulators of Notch signaling, optionally wherein said liver bud progenitors are obtained from the method of claim 6.

41.-44. (canceled)

45. A method of maintaining hepatocytes or hepatocyte-like cells in a perivenous state comprising contacting said cells with one or more inhibitors of Notch signaling and/or one or more activators of Wnt signaling, optionally further comprising contacting said hepatocytes or hepatocyte-like cells with: one or more inhibitors of TGFb; and/or one or more activators of estrogen signaling; one or more inhibitors of cyclic AMP/PKA signaling, optionally wherein said cells are obtained from the method of claim 24, optionally wherein said hepatocytes or hepatocyte-like cells are obtained from a human liver.

46.-48. (canceled)

49. A method of maintaining hepatocytes in a periportal state comprising contacting said cells with one or more activators of cyclic AMP/PKA signaling, optionally wherein said hepatocytes are obtained from a human liver.

50. (canceled)

51. A kit for differentiating
(a) cells of the definitive endoderm (DE) lineage into posterior foregut lineage comprising one or more of the following factors: one or more retinoic acid activators; and/or one or more inhibitors of TGFβ signaling; or
(b) cells of the posterior foregut lineage into liver bud progenitors comprising one or more of the following factors: one or more activators of TGFβ signaling one or more modulators of Wnt signaling; and/or one or more activators of cyclic AMP/PKA signaling; or
(c) liver bud progenitors into hepatic progenitors comprising one or more of the following factors: one or more inhibitors of TGFβ signaling one or more inhibitors of FGF signaling; and/or one or more inhibitors of Notch signaling; or
(d) liver bud progenitors into hepatic progenitors comprising one or more of the following factors: one or more inhibitors of Notch signaling; activators of ascorbic acid signaling; one or more activators of cyclic AMP/PKA signaling; and/or one or more activators of insulin signaling; or
(e) hepatic progenitors into perivenous hepatocyte-like cells comprising one or more of the following factors: one or more inhibitors of Notch signaling; one or more activators of glucocorticoid signaling; one or more activators of insulin signaling; one or more activators of ascorbic acid signaling and/or one or more activators of TGFβ signaling; or
(f) hepatic progenitors into hepatocytes or hepatocyte-like cells comprising one or more of the following factors: one or more activators of cyclic AMP/PKA signaling; one or more activators of glucocorticoid signaling; one or more activators of insulin signaling one or more activators of ascorbic acid signaling; and/or one or more activators of Wnt signaling one or more inhibitors of Notch signaling.

52.-56. (canceled)

57. A kit for maintaining
(a) liver bud progenitors in a self-renewal state comprising one or more of the following factors: one or more activators of FGF signaling; and/or one or more activators of Wnt signaling, optionally further comprising one or more of the following factors; one or more activators of BMP signaling; one or more epidermal growth factors; one or more inhibitors of TGFβ signaling; and/or one or more inhibitors of Notch signaling; or
(b) hepatocytes or hepatocyte-like cells in a perivenous state comprising one or more of the following factors: one or more inhibitors of Notch signaling and/or one or more activators of Wnt signaling, optionally further comprising one or more of the following factors: one or more inhibitors of TGFb; and/or one or more activators of estrogen signaling; one or more inhibitors of cyclic AMP/PKA signaling; or
(c) hepatocytes or hepatocyte-like cells in a periportal state comprising one or more activators of cyclic AMP/PKA signaling.

58.-61. (canceled)

62. A surface marker for isolating or selecting for LB cells selected from EGFR or CD99; or a surface marker for isolating or selecting for MHG cells comprising CD325 (N-cadherin).

63. (canceled)

64. A method of screening for a cellular response, the method comprising: a) contacting a population of cells generated according to any one of claims 1, 6, 14, 24, 33, 40, 45, 49, 51, 57, or 62 with a pharmacological agent; and b) evaluating the population of cells for a cellular response induced by the pharmacological agent, optionally wherein the screening is in vitro screening and the contacting is performed in vitro, optionally wherein the screening is in vivo screening and the contacting is performed by administering the pharmacological agent to a host animal that comprises the population of cells.

65. (canceled)

66. (canceled)

67. A method of screening for a phenotype, the method comprising: a) administering to a host animal a population of cells generated according to any one of claims 1, 6, 14, 24, 33, 40, 45, 49, 51, 57, or 62 wherein the cells of the population of cells comprise a genetic modification in at least one genetic locus; and b) evaluating the host animal for a detectable phenotype induced by the administered population of cells, optionally wherein the genetic modification in at least one genetic locus results in the disruption or deletion of at least one gene, optionally wherein the population of cells comprise liver cells and the detectable phenotype comprises a survival enhancement.

68. (canceled)

69. (canceled)

70. A method of treating a subject for a condition, the method comprising: a) administering the subject a therapeutically effective amount of cells generated according to any one of claims 1, 6, 14, 24, 33, 40, 45, 49, 51, 57, or 62 in order to treat the subject for the condition, optionally wherein the cells are co-administered with at least one pro-survival or pro-engraftment factor, optionally wherein the cells comprise a genetic modification in at least one genetic locus.

71. (canceled)

72. (canceled)
